(19) United States
(12) Patent Application Publication
(10) Pub. No.: US 2009/0312586 A1
(43) Pub. Date: Dec. 17, 2009

(54) HYDROGENATION OF MULTI-BROMINATED ALKANES

(75) Inventors: John J. Waycuilis, Cypress, TX (US); William J. Turner, Seabrook, TX (US)

Correspondence Address: MARATHON OIL COMPANY;C/O LAW OFFICE OF JACK E. EBEL, 165 SOUTH UNION BOULEVARD, SUITE 902, LAKEWOOD, CO, 80228, US

(73) Assignee: MARATHON GTF TECHNOLOGY, LTD., Houston, TX (US)

(21) Appl. No.: 12/139,135

(22) Filed: Jun. 13, 2008

(51) Int. Cl. C07C 17/013; C07C 17/25; B01J 19/02

(52) U.S. Cl. 570/227; 570/231; 422/189

(57) ABSTRACT

Methods and systems for the hydrogenation of multi-brominated alkanes are provided herein. An embodiment of the present invention comprises a method, the method comprising: reacting at least hydrogen and multi-brominated alkanes in the presence of a catalyst to form a hydrogenated stream comprising brominated alkanes having fewer bromine substituents than the multi-brominated alkanes reacted with the hydrogen. Embodiments of the method further may comprise forming brominated alkanes. Embodiments of the method further may comprising forming product hydrocarbons from brominated alkanes.

### BACKGROUND

The present invention relates to hydrogenation of multi-brominated alkanes and, more particularly, in one or more embodiments, to a method and system wherein mono-brominated alkanes are formed by contacting a stream comprising multi-brominated alkanes with hydrogen.

Mono-halogenated alkanes may used in the production of a variety of desirable products, including, but not limited to, alcohols, ethers, olefins, and higher hydrocarbons, such as C3, C4, and C5+ gasoline-range and heavier hydrocarbons. For instance, mono-halogenated alkanes may be converted to corresponding alcohols over a metal oxide. In another instance, mono-brominated alkanes may be converted to higher molecular weight hydrocarbons over an appropriate catalyst.

To produce mono-halogenated alkanes, alkanes may be brominated with a source of bromine. In one instance, a gaseous feed comprising lower molecular weight alkanes may be reacted with bromine vapor to form brominated alkanes. While the bromination of alkanes may be reasonably selective with respect to mono-brominated alkanes, a significant amount of multi-brominated alkanes also may be produced. For instance, in the case of the non-catalyzed bromination of methane operated with excess methane in the range of about 4:1 to about 9:1, the reaction selectivity generally may be in the range of about 70% to about 80% mono-brominated methane and about 20% to about 30% di-brominated methane. Depending on the application, however, the multi-brominated alkanes (such as the di-brominated methane) may be a less desirable byproduct. By way of example, di-brominated methane may be undesirable in a subsequent hydrocarbon synthesis reaction, in that the presence of di-brominated methane may promote coke formation and deactivate the synthesis catalyst.

To improve the selectivity with respect to mono-brominated alkanes, the bromination reaction may be run with a larger excess of alkanes. However, increasing the amount of alkanes dilutes the products and reactants in the system, potentially requiring the recycling of larger amounts of methane and other light alkanes within the system, which may result in increased power and processing costs due, for example, to the increased size of vessels and piping needed to handle the larger amounts of alkanes. In another instance, multi-brominated alkanes (such as di-brominated methane) may be reacted with light alkanes (such as C2-C4 alkanes which may be more reactive than methane) to form mono-brominated alkanes. However, the reaction of di- and tri-brominated alkanes with light alkanes is generally kinetically slow, requiring long residence times of up to a minute or longer and not highly selective to mono-brominated alkanes (such as mono-brominated methane and mono-brominated ethane), and some coking possibly due to free-radical chain reactions also may occur, limiting the efficiency of carbon conversion to useful products.

### SUMMARY

The present invention relates to hydrogenation of multi-brominated alkanes and, more particularly, in one or more embodiments, to a method and system wherein mono-brominated alkanes are formed by contacting a stream comprising multi-brominated alkanes with hydrogen.

An embodiment of the present invention comprises a method, the method comprising: reacting at least hydrogen and multi-brominated alkanes in the presence of a catalyst to form a hydrogenated stream comprising brominated alkanes having fewer bromine substituents than the multi-brominated alkanes reacted with the hydrogen.

Another embodiment of the present invention comprises a method, the method comprising: forming bromination products comprising brominated alkanes from bromination reactants comprising alkanes and bromine, wherein the brominated alkanes comprise mono-brominated alkanes and multi-brominated alkanes; forming hydrogenation products comprising additional mono-brominated alkanes from hydrogenation reactants comprising hydrogen and at least a portion of the multi-brominated alkanes formed from the bromination reactants; and forming synthesis products comprising hydrocarbons from synthesis reactants comprising reactant mono-brominated bromines, wherein the reactant mono-brominated bromines comprise at least a portion of the mono-brominated alkanes formed from the bromination reactants and at least a portion of the additional mono-brominated alkanes formed from the hydrogenation reactants.

Another embodiment of the present invention comprises a system, the system comprising: a bromination reactor configured to form bromination products comprising brominated alkanes from bromination reactants comprising alkanes and bromine, wherein the brominated alkanes comprise mono-brominated alkanes and multi-brominated alkanes; a hydrogenation reactor in fluid communication with the bromination reactor and configured to form hydrogenation products comprising additional mono-brominated alkanes from hydrogenation reactants comprising hydrogen and at least a portion of the multi-brominated alkanes from the bromination reactor; and a synthesis reactor in fluid communication with the hydrogenation reactor and configured to form synthesis products comprising hydrocarbons from synthesis reactants comprising reactant mono-brominated bromines, wherein the reactant mono-brominated bromines comprise at least a portion of the mono-brominated alkanes from the bromination reactor and at least a portion of the additional mono-brominated alkanes from the hydrogenation reactor.

The features and advantages of the present invention will be readily apparent to those skilled in the art. While numerous changes may be made by those skilled in the art, such changes are within the spirit of the invention.

### BRIEF DESCRIPTION OF THE DRAWINGS

These drawings illustrate certain aspects of some of the embodiments of the present invention, and should not be used to limit or define the invention.

FIG. 1 is an example block diagram of a process for the hydrogenation of multi-brominated alkanes, in accordance with one embodiment of the present invention.

FIG. 2 is an example block diagram of a process for the hydrogenation of multi-brominated alkanes that includes bromination, in accordance with one embodiment of the present invention.

FIG. 3 is an example block diagram of a process for the production of product hydrocarbons that includes bromination and hydrogenation, in accordance with one embodiment of the present invention.

FIG. 4 is another example block diagram of a process for the production of product hydrocarbons that includes bromination and hydrogenation, in accordance with one embodiment of the present invention.

FIG. 5 is another example block diagram of a process for the production of product hydrocarbons that includes bromination and hydrogenation, in accordance with one embodiment of the present invention.

FIG. 6 is another example block diagram of a process for the production of product hydrocarbons that includes bromination and hydrogenation, wherein hydrogen for hydrogenation is produced via steam-methane reforming, in accordance with one embodiment of the present invention.

FIG. 7 is another example block diagram of a process for the production of product hydrocarbons that includes bromination and hydrogenation, wherein hydrogen for hydrogenation is produced via electrolysis, in accordance with one embodiment of the present invention.

FIG. 8 is another example block diagram of a process for the production of product hydrocarbons that includes bromination and hydrogenation, wherein hydrogen for hydrogenation is produced via electrolysis, in accordance with one embodiment of the present invention.

FIG. 9 is another example block diagram of a process for the production of product hydrocarbons that includes bromination and hydrogenation, wherein hydrogen for hydrogenation is produced via electrolysis, in accordance with one embodiment of the present invention.

FIGS. 10-14 are additional example block diagrams of processes for the production of product hydrocarbons that include hydrogenation and bromination, wherein mono-brominated alkanes bypass the hydrogenation, in accordance with embodiments of the present invention.

FIG. 15 is a graph of conversion of di-brominated methane versus time during hydrogenation, in accordance with one embodiment of the present invention.

FIG. 16 is a graph of concentration of di-brominated methane and mono-brominated methane entering and exiting a hydrogenation reactor during hydrogenation, in accordance with one embodiment of the present invention.

FIG. 17 is a graph of concentration of hydrogen and hydrogen bromide entering and exiting a hydrogenation reactor during hydrogenation, in accordance with one embodiment of the present invention.

FIG. 18 is a graph of conversion of di-brominated methane versus time during hydrogenation, in accordance with one embodiment of the present invention.

FIG. 19 is a graph of concentration of di-brominated methane and mono-brominated methane entering and exiting a hydrogenation reactor during hydrogenation, in accordance with one embodiment of the present invention.

FIG. 20 is a graph of concentration of hydrogen and hydrogen bromide entering and exiting a hydrogenation reactor during hydrogenation, in accordance with one embodiment of the present invention.

### DESCRIPTION OF PREFERRED EMBODIMENTS

The present invention relates to hydrogenation of multi-brominated alkanes and, more particularly, in one or more embodiments, to a method and system wherein mono-brominated alkanes are formed by contacting a stream comprising multi-brominated alkanes with hydrogen.

There may be many potential advantages to the methods and systems of the present invention, only some of which are alluded to herein. One of the many potential advantages may be that hydrogenation of multi-brominated alkanes should increase the amount of mono-halogenated alkanes formed. Accordingly, techniques wherein higher proportions of mono-brominated alkanes are desired may also be improved. For example, the efficiency of carbon conversion to useful products may be improved due to the improved selectivity with respect to mono-brominated alkanes, such as in the conversion of the brominated alkanes to product hydrocarbons. Among other things, higher proportions of the mono-brominated alkanes may improve the efficiency of carbon conversion, for example, due to reduced formation of coke and slower deactivation of the catalyst.

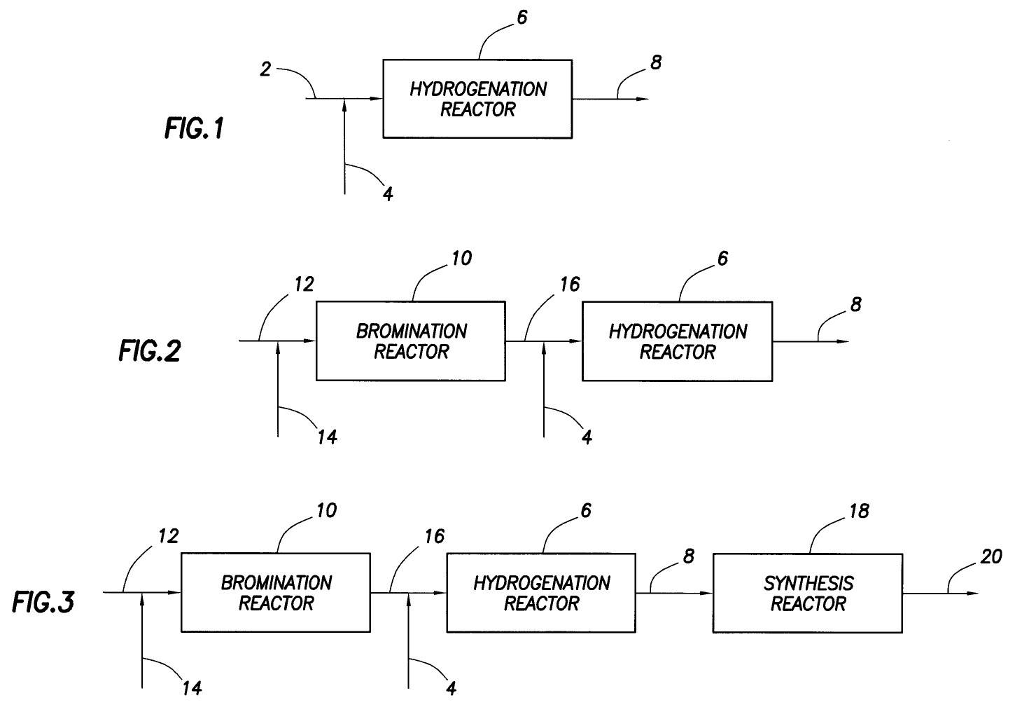

Referring to FIG. 1, an example block diagram of a process for the hydrogenation of multi-brominated alkanes is illustrated, in accordance with one embodiment of the present invention. In the illustrated embodiment, hydrogenation feed stream 2 comprising multi-brominated alkanes may be combined with hydrogen stream 4 and introduced into hydrogenation reactor 6. In hydrogenation reactor 6, the multi-brominated alkanes react with hydrogen to form hydrogen bromide and one or more brominated alkanes with fewer bromine substituents. While FIG. 1 illustrates the combination of hydrogenation feed stream 2 and hydrogen stream 4 prior to hydrogenation reactor 6, those of ordinary skill in the art should appreciate that these streams may be combined in the reactor. By way of example, hydrogenation feed stream 2 and hydrogen stream 4 may be introduced into hydrogenation reactor 6 such that they mix prior to contacting a catalyst, if any, present in the reactor.

Hydrogenation feed stream 2 generally comprises multi-brominated alkanes and may be at a pressure, for example, in the range of about 1 atm to about 100 atm and, alternatively, of about 1 atm to 30 atm. The alkanes may include, for example, lower molecular weight alkanes. As used herein, the term “lower molecular weight alkanes” refers to methane, ethane, propane, butane, pentane, or mixtures thereof. In certain embodiments, the lower molecular weight alkanes may be methane. The multi-brominated alkanes may include di-brominated alkanes, tri-brominated alkanes, tetra-brominated alkanes, or mixtures thereof. In certain embodiments, hydrogenation feed stream 2 also may comprise mono-brominated alkanes, hydrogen bromide, or combinations thereof.

Hydrogen stream 4 generally comprises hydrogen and may be at a pressure, for example, in the range of about 1 atm to about 100 atm and, alternatively, of about 1 atm to 30 atm. As will be discussed in more detail below, the hydrogen present in hydrogen stream 4 may be provided via any suitable source, including steam-methane reforming, the water-gas shift reaction of carbon monoxide, or electrolysis of water, metal halide salt, or hydrogen bromide. Because embodiments described below produce hydrogen bromide, electrolysis of the hydrogen bromide may be a particularly suitable technique for the production of hydrogen in certain embodiments of the present invention. It is believed that the electrolysis of the hydrogen bromide also may be less energy intensive than steam-methane reforming. In certain embodiments, the mole ratio of the hydrogen (H₂) to the multi-brominated alkanes in the mixture introduced to hydrogenation reactor 6 may be, for example, at least about 1:1. For example, the mixture introduced into the hydrogenation reactor 2 may have a hydrogen (H₂) to di-brominated methane mole ratio of about 1:1.

In hydrogenation reactor 6, the multi-brominated alkanes may react with the hydrogen to form hydrogen bromide and one or more brominated alkanes with fewer bromine substituents with respect to the multi-brominated alkanes. For example, di-brominated alkanes may react with the hydrogen to form mono-brominated alkanes. In the case of di-brominated methane, the reaction with hydrogen may occur in accordance with the following general reaction:

CH²Br²+H²→CH³Br+HBr (1)

In accordance with embodiments of the present invention, it is believed that hydrogenation reactor 6 may be operated to form mono-brominated alkanes and hydrogen bromide with a high, up to 100%, selectivity, in that up to 100% of the multi-brominated alkanes may be converted to mono-brominated alkanes. However, some small amount of coking should generally occur, such that a gradual deactivation of the catalyst occurs. It is believed that higher temperature, while resulting in high apparent conversion of the multi-brominated alkanes, also accelerates coking. Thus, operation at lower temperatures, at the expense of requiring a larger reactor to achieve high conversion of the multi-brominated alkanes, may be acceptable due to the lower losses due to the formation of coke and slower catalyst deactivation. It has been found that high activity may be restored to the catalyst be regeneration with an oxygen-containing gas mixture or air.

The reaction in hydrogenation reactor 6 between the multi-brominated alkanes and the hydrogen may be a homogeneous gas-phase reaction or a heterogeneous catalytic reaction thereof, in accordance with embodiments of the present invention. While the reaction in hydrogenation reactor 6 may occur, for example, at temperatures in the range of about 150° C. to about 650° C. and pressures in the range of about 1 atm to about 100 atm and, alternatively, of about 1 atm to 30 atm, those of ordinary skill in the art, with the benefit of this disclosure, should appreciate that the homogeneous gas-phase reaction may occur at higher temperatures. In certain embodiments, the multi-brominated alkanes and the hydrogen may be reacted at temperatures in the range of about 300° C. to about 650° C.

As mentioned in the preceding paragraph, the reaction in the hydrogenation reactor 6 may be conducted catalytically. Examples of suitable catalysts for hydrogenation reactor 6 include, but are not limited to, metals capable of forming one or more thermally reversible complexes with bromine. In certain embodiments, suitable catalysts include, but are not limited to, metals with more than one oxidation state capable of forming multiple thermally reversible complexes with the bromine. Specific examples of suitable catalysts that form multiple thermally reversible complexes with bromine may include, but are not limited to, iron, copper, tungsten, molybdenum, vanadium, chromium, platinum, and palladium. Examples of suitable catalysts that have only one oxidation state and form a single complex with bromine and are believed to also have some activity may include, but are not limited to, nickel, cobalt, zinc, magnesium, calcium, and aluminum. In certain embodiments, the metals may be promoted, for example, with Cu or other transition metals. Additional examples of suitable catalysts include metal halide salts with Lewis-acid functionality and metal oxy halides. In certain embodiments, the catalyst may include an oxide or bromide of the metal deposited on a support. For example, a metal may be deposited as a bromide (e.g., iron bromide) or an oxide (e.g., iron oxide) on an inert support, such as silica, alumina, and the like. By way of further example, a metal (e.g., platinum) may be dispersed on an inert support, such as low-surface area silica support.

Hydrogenated stream 8 comprising the brominated alkane with fewer bromine substituents may be withdrawn from hydrogenation reactor 6. By way of example, hydrogenated stream 8 withdrawn from the hydrogenation reactor may comprise mono-brominated alkanes produced in hydrogenation reactor 6.

Referring to FIG. 2, an example block diagram of a process that includes bromination and hydrogenation of multi-brominated alkanes is illustrated, in accordance with one embodiment of the present invention. In the illustrated embodiment, the process includes bromination reactor 10 and hydrogenation reactor 6. As illustrated, gaseous feed stream 12 comprising alkanes may be combined with bromine stream 14, and the resulting mixture may be introduced into bromination reactor 10. While FIG. 2 illustrates the combination of gaseous feed stream 12 and bromine stream 14 prior to bromination reactor 10, those of ordinary skill in the art, with the benefit of this disclosure, should appreciate that gaseous feed stream 12 and bromine stream 14 may be combined in bromination reactor 10.

Gaseous feed stream 12 generally comprises alkanes and may be at a pressure, for example, in the range of about 1 atm to about 100 atm and, alternatively, of about 1 atm to about 30 atm. The alkanes present in the gaseous feed stream may include, for example, lower molecular weight alkanes. As previously mentioned, in certain embodiments, the lower molecular weight alkanes may be methane. Also, gaseous feed stream 12 used in embodiments of the present invention may be any source of gas containing lower molecular weight alkanes whether naturally occurring or synthetically produced. Examples of suitable gaseous feeds that may used in embodiments of the process of the present invention include, but are not limited to, natural gas, coalbed methane, regasified liquefied natural gas, gas derived from gas hydrates, chlathrates or both, gas derived from anaerobic decomposition of organic matter or biomass, synthetically produced natural gas or alkanes, and mixtures thereof. In certain embodiments, gaseous feed stream 12 may include a feed gas plus a recycled gas stream. In certain embodiments, gaseous feed stream 12 may be treated to remove sulfur compounds and carbon dioxide. In any event, in certain embodiments, small amounts of carbon dioxide, e.g. less than about 2 mol %, may be present in gaseous feed stream 12.

Bromine stream 14 generally comprises bromine and may be at a pressure, for example, in the range of about 1 atm to about 100 atm and, alternatively, of about 1 atm to about 30 atm. In certain embodiments, the bromine may be dry, in that it is substantially free of water vapor. In certain embodiments, the bromine present in bromine stream 14 may be in a gaseous state, a liquid state, or a combination thereof. While not illustrated, in certain embodiments, bromine stream 14 may contain recycled bromine that is recovered in the process as well as make-up bromine that is introduced into the process. While also not illustrated, in certain embodiments, the mixture of the gaseous feed stream and the bromine may be passed to a heat exchanger for evaporation of the bromine prior to introduction into bromination reactor 10.

As previously mentioned, gaseous feed stream 12 and bromine stream 14 may be combined and introduced into bromination reactor 10. The mole ratio of the alkanes in gaseous feed stream 12 to the bromine in bromine stream 14 may be, for example, in excess of 2.5:1. While not illustrated, in certain embodiments, bromination reactor 10 may have an inlet pre-heater zone for heating the mixture of the alkanes and bromine to a reaction initiation temperature, for example, in the range of about 250° C. to about 400° C.

In bromination reactor 10, the alkanes may be reacted with the bromine to form brominated alkanes and hydrogen bromide. By way of example, methane may react in bromination reactor 10 with bromine to form brominated methane and hydrogen bromide. In the case of methane reacting with bromine, the formation of mono-brominated methane occurs in accordance with the following general reaction:

CH⁴+Br²→CH³Br+HBr (2)

Due to the free-radical mechanism of the gas-phase bromination reaction, multi-brominated alkanes may also be formed in bromination reactor 10. In certain embodiments, about 10% to about 30% mole fraction of the brominated alkanes formed in bromination reactor 10 may be multi-brominated alkanes. For example, in the case of the bromination of methane, at a methane-to-bromine ratio of about 6:1 the selectivity to the mono-brominated methyl bromide may average approximately 88%, depending on reaction conditions such as residence time, temperature, turbulent mixing, etc. At these conditions, di-brominated methane and only very small amounts of tri-brominated methane and other brominated alkanes should also be formed in the bromination reaction. By way of example, if a lower methane-to-bromine ratio of approximately 2.6 to 1 is used, selectivity to the mono-brominated methane may fall to the range of about 65% to about 75% depending on other reaction conditions. If a methane-to-bromine ratio significantly less than about 2.5 to 1 is used, even lower selectivity to mono-brominated methane occurs, and, moreover, significant formation of undesirable carbon soot is observed. Higher alkanes, such as ethane, propane, and butane, may also be readily brominated resulting in mono- and multi-brominated alkanes, such as brominated ethane, brominated propane, and brominated butane.

In certain embodiments, the bromination reaction in bromination reactor 10 occurs exothermically, for example, at a temperature in the range of about 250° C. to about 600° C. and at a pressure in the range of about 1 atm to about 100 atm and, alternatively, of about 1 atm to about 30 atm. The upper limit of this temperature range may be greater than the upper limit of the reaction initiation temperature range to which the feed mixture may be heated due to the exothermic nature of the bromination reaction. As will be appreciated by those of ordinary skill in the art, with the benefit of this disclosure, the reaction in bromination reactor 10 may be a homogeneous gas phase reaction or a heterogeneous catalytic reaction. Examples of suitable catalysts that may be used in bromination reactor 10 include, but are not limited to, platinum, palladium, or supported non-stoichiometric metal oxy-halides such as FeOₓBr_{y} or FeOₓCl_{y} or supported stoichiometric metal oxy-halides such as TaOF3, NbOF3, ZrOF2, SbOF3 as described in Olah, et al, J. Am. Chem. Soc. 1985, 107, 7097-7105. Although use of such catalysts may allow selective mono-bromination at lower temperatures in the range of about 200° C. to 250° C., conversion rates are typically low at these lower temperatures; whereas at higher temperatures selectivity is less with more multi-brominates alkanes being formed.

As set forth above, the bromine fed into bromination reactor 10 may be dry, in certain embodiments of the present invention. Elimination of substantially all water vapor from the bromination reaction in bromination reactor 10 substantially eliminates the formation of unwanted carbon dioxide, thereby increasing the selectivity of the alkane bromination to brominated alkanes and potentially eliminating the large amount of waste heat generated in the formation of carbon dioxide from alkanes. Further, elimination of substantially all water vapor should minimize hydrothermal degradation of downstream catalysts that may be used, in certain embodiments of the present invention.

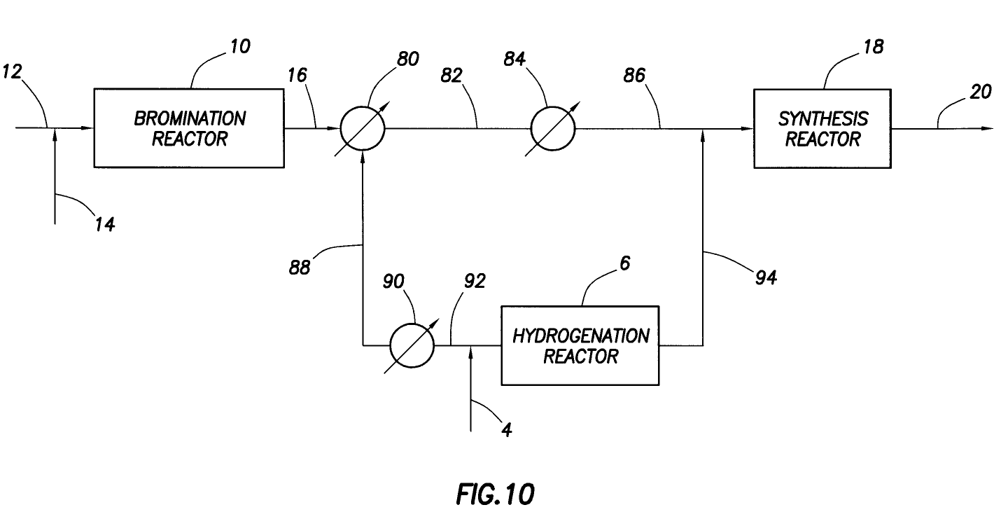

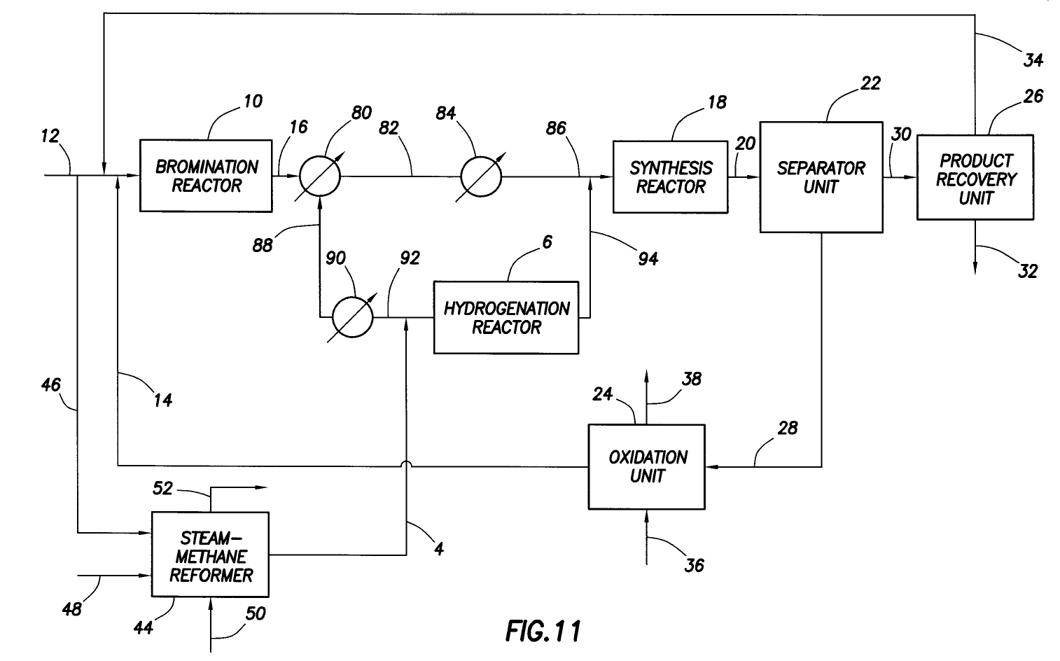

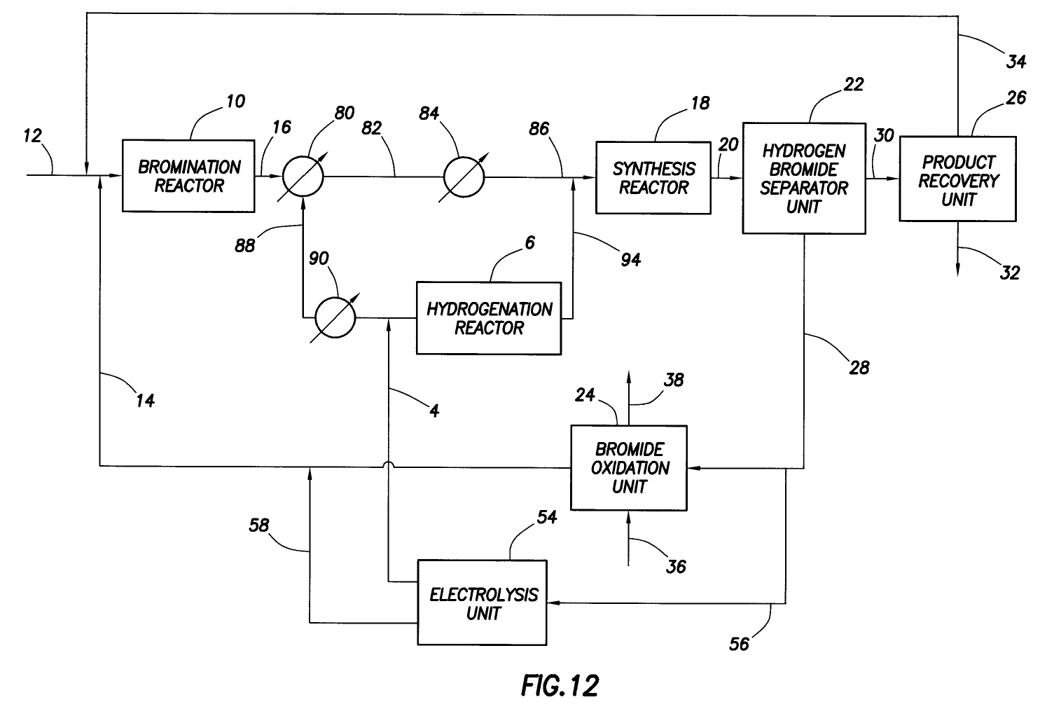

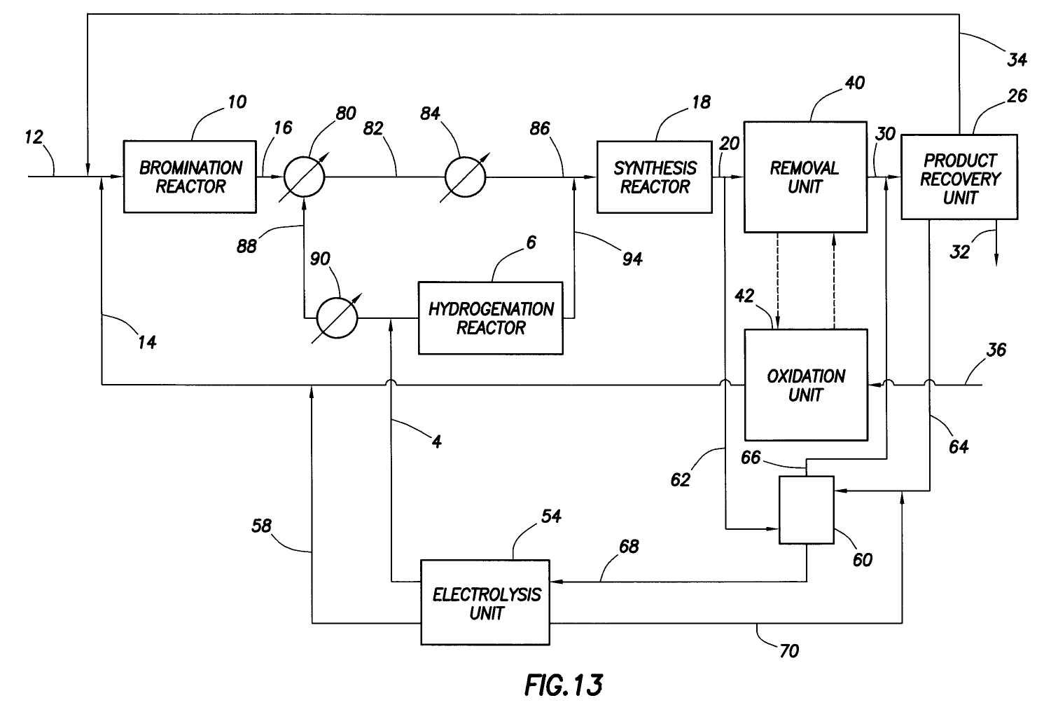

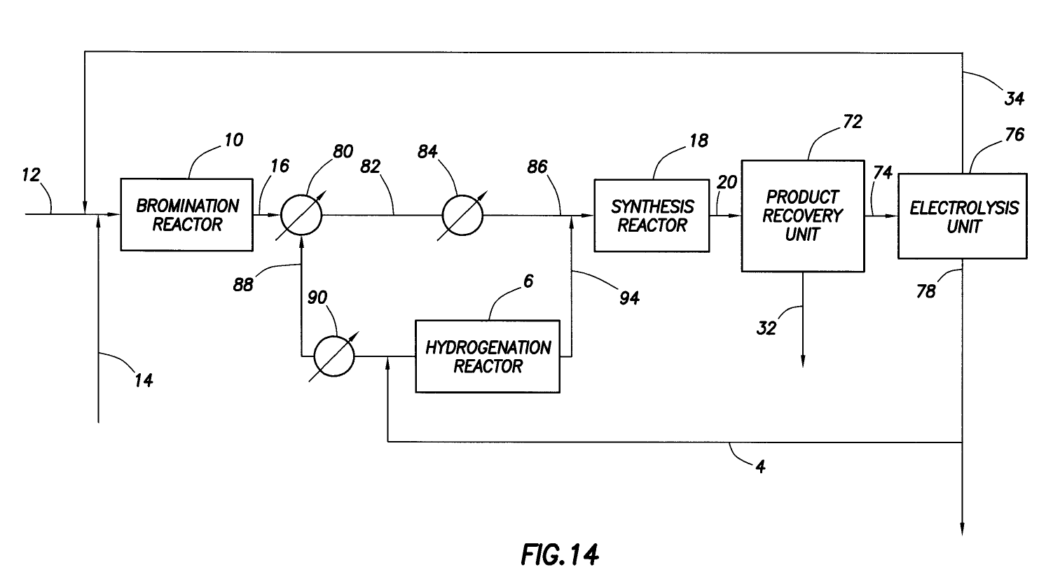

As illustrated in FIG. 2, brominated stream 16 may be withdrawn from bromination reactor 10. In general, brominated stream 16 withdrawn from bromination reactor 10 comprises brominated alkanes and hydrogen bromide. The brominated alkanes present in brominated stream 16 may comprise mono- and multi-brominated alkanes. In the illustrated embodiment, brominated stream 16 may be combined with hydrogen stream 4 and introduced into hydrogenation reactor 6. As will be discussed in more detail below with respect to FIGS. 10-14, a large portion of the mono-brominated alkanes and hydrogen bromide present in brominated stream 16 may bypass hydrogenation reactor 6, in certain embodiments, so that hydrogenation stream reactor 6 is fed a more concentrated stream of reactants.

In hydrogenation reactor 6, the multi-brominated alkanes present in brominated stream 16 may be reacted with the hydrogen to form hydrogen bromide and one or more brominated alkanes with fewer bromine substituents. In accordance with embodiments of the present invention, it is believed that hydrogenation reactor 6 may be operated to form mono-brominated alkanes and hydrogen bromide with a high, up to 100%, selectivity, in that up to nearly 100% of the multi-brominated alkanes may be converted to mono-brominated alkanes. However, some small amount of coking should generally occur, such that a gradual deactivation of the catalyst occurs. It is believed that higher temperature, while resulting in high apparent conversion of the multi-brominated alkanes, also accelerates coking. Thus, operation at lower temperatures, at the expense of requiring a larger reactor to achieve high conversion of the multi-brominated alkanes, may be acceptable due to the lower losses due to the formation of coke and slower catalyst deactivation. It has been found that high activity may be restored to the catalyst be regeneration with an oxygen-containing gas mixture or air. Hydrogenation reactor 6 and hydrogen stream 4 are described in more detail with respect to FIG. 1 above.

Hydrogenated stream 8 comprising the brominated alkanes with fewer bromine substituents may be withdrawn from hydrogenation reactor 6. By way of example, hydrogenated stream 8 withdrawn from hydrogenation reactor 6 may comprise mono-brominated alkanes produced in hydrogenation reactor 6. Hydrogenated stream 8 also may comprise mono-brominated alkanes and hydrogen bromide that were produced in bromination reactor 10.

In accordance with embodiments of the present invention, the process described above with respect to FIGS. 1 and 2 for the hydrogenation of multi-brominated alkanes may be used in a process for the production of product hydrocarbons over a dehydrohalogenation/oligomerization catalyst. The product hydrocarbons generally may include, for example, C3, C4, and C5+ gasoline-range and heavier hydrocarbons, including, for example, branched alkanes, substituted aromatics, napthenes, or olefins, such as ethylene, propylene, and the like. Due to the increased formation of mono-brominated alkanes, processes for the production of the product hydrocarbons may be improved, in that the efficiency of the carbon conversion to useful products may be improved, for example, due to reduced formation of coke and slower deactivation of the catalyst.

Referring to FIG. 3, an example block diagram of a process for the production of product hydrocarbons that includes bromination and hydrogenation is illustrated, in accordance with one embodiment of the present invention. In the illustrated embodiment, the process includes bromination reactor 10, hydrogenation reactor 6, and synthesis reactor 18. Example processes for the production of product hydrocarbons that include bromination followed by a synthesis reaction are described in more detail in U.S. Pat. No. 7,244,867, U.S. Pat. No. 7,348,464, and U.S. Patent Pub. No. 2006/0100469, the entire disclosures of which are incorporated herein by reference.

As illustrated in FIG. 3, gaseous feed stream 12 comprising alkanes may be combined with bromine stream 14 and the resulting mixture may be introduced into bromination reactor 10. In bromination reactor 10, the alkanes may be reacted with the bromine to form brominated alkanes and hydrogen bromide. Gaseous feed stream 12, bromine stream 14, and bromination reactor 10 are described in more detail above with respect to FIG. 2.

Brominated stream 16 may be withdrawn from bromination reactor 10. In general, brominated stream 16 withdrawn from bromination reactor 10 comprises brominated alkanes and hydrogen bromide. The brominated alkanes present in brominated stream 16 may comprise mono- and multi-brominated alkanes. In the illustrated embodiment, brominated stream 16 may be combined with hydrogen stream 4 and introduced into hydrogenation reactor 6. In hydrogenation reactor 6, the multi-brominated alkanes present in brominated stream 16 may react with the hydrogen to form hydrogen bromide and one or more brominated alkanes with fewer bromine substituents. In accordance with embodiments of the present invention, it is believed that hydrogenation reactor 6 may be operated to form mono-brominated alkanes and hydrogen bromide with a high, up to 100%, selectivity, in that up to nearly 100% of the multi-brominated alkanes may be converted to mono-brominated alkanes. Hydrogenation reactor 6 and hydrogen stream 4 are described in more detail above with respect to FIG. 1.

Hydrogenated stream 8 comprising the brominated alkanes with fewer bromine substituents may be withdrawn from hydrogenation reactor 6 and introduced into synthesis reactor 18. By way of example, hydrogenated stream 8 withdrawn from hydrogenation reactor 6 may comprise a mono-brominated alkane produced in hydrogenation reactor 6. Hydrogenation stream 8 also may comprise mono-brominated alkanes and hydrogen bromide that were produced in bromination reactor 10. While not illustrated, hydrogenated stream 8 may be cooled in a heat exchanger to a temperature in the range of about 150° C. to about 450° C. before being introduced to synthesis reactor 18, to allow for the temperature rise due to the exothermic synthesis reaction. In synthesis reactor 18, the brominated alkanes may be reacted exothermically in the presence of a catalyst to form the product hydrocarbons and additional hydrogen bromide. The reaction may occur, for example, at a temperature in the range of about 150° C. to about 500° C. and a pressure in the range of about 1 atm to 100 atm and, alternatively, of about 1 atm to about 30 atm.

The catalyst may be any of a variety of suitable materials for catalyzing the conversion of the brominated alkanes to product hydrocarbons. In certain embodiments, synthesis reactor 18 may comprise a fixed bed of the catalyst. A fluidized-bed of synthesis catalyst may also be used in certain circumstances, particularly in larger applications and may have certain advantages, such as constant removal of coke and a steady selectivity to product composition. Examples of suitable catalysts include a fairly wide range of materials that have the common functionality of being acidic ion-exchangers and which also contain a synthetic crystalline alumino-silicate oxide framework. In certain embodiments, a portion of the aluminum in the crystalline alumino-silicate oxide framework may be substituted with magnesium, boron, gallium and/or titanium. In certain embodiments, a portion of the silicon in the crystalline alumino-silicate oxide framework may be optionally substituted with phosphorus. The crystalline alumino-silicate catalyst generally may have a significant anionic charge within the crystalline alumino-silicate oxide framework structure which may be balanced, for example, by cations of elements selected from the group H, Li, Na, K or Cs or the group Mg, Ca, Sr or Ba. Although zeolitic catalysts may be commonly obtained in a sodium form, a protonic or hydrogen form (via ion-exchange with ammonium hydroxide, and subsequent calcining) is preferred, or a mixed protonic/sodium form may also be used. The zeolite may also be modified by ion exchange with other alkali metal cations, such as Li, K, or Cs, with alkali-earth metal cations, such as Mg, Ca, Sr, or Ba, or with transition metal cations, such as Ni, Mn, V, and W. Such subsequent ion-exchange, may replace the charge-balancing counter-ions, but furthermore may also partially replace ions in the oxide framework resulting in a modification of the crystalline make-up and structure of the oxide framework. The crystalline alumino-silicate or substituted crystalline alumino-silicate may include a microporous or mesoporous crystalline aluminosilicate, but, in certain embodiments, may include a synthetic microporous crystalline zeolite, and, for example, being of the MFI structure such as ZSM-5. Moreover, the crystalline alumino-silicate or substituted crystalline alumino-silicate, in certain embodiments, may be subsequently impregnated with an aqueous solution of a Mg, Ca, Sr, or Ba salt. In certain embodiments, the salts may be a halide salt, such as a bromide salt, such as MgBr₂. Optionally, the crystalline alumino-silicate or substituted crystalline alumino-silicate may also contain between about 0.1 to about 1 weight % Pt, about 0.1 to 5 weight % Pd, or about 0.1 to about 5 weight % Ni in the metallic state. Although, such materials are primarily initially crystalline, it should be noted that some crystalline catalysts may undergo some loss of crystallinity either due to initial ion-exchange or impregnation or due to operation at the reaction conditions or during regeneration and hence my also contain significant amorphous character, yet still retain significant, and in some cases improved activity.

The particular catalyst used in synthesis reactor 18 will depend, for example, upon the particular product hydrocarbons that are desired. For example, when product hydrocarbons having primarily C3, C4 and C5+ gasoline-range aromatic compounds and heavier hydrocarbon fractions are desired, a ZSM-5 zeolite catalyst may be used. When it is desired to produce product hydrocarbons comprising a mixture of olefins and C₅+ products, an X-type or Y-type zeolite catalyst or SAPO zeolite catalyst may be used. Examples of suitable zeolites include an X-type, such as 10-X, or Y-type zeolite, although other zeolites with differing pore sizes and acidities, may be used in embodiments of the present invention.

The temperature at which synthesis reactor 18 is operated is one parameter in determining the selectivity of the reaction to the particular products hydrocarbons that are desired. Where, for example, an X-type or Y-type zeolite or SAPO zeolite catalyst is used and it is desired to produce olefins, synthesis reactor 18 may be operated at a temperature within the range of about 250° C. to about 500° C. Temperatures above about 450° C. in synthesis reactor 18 may result in increased yields of light hydrocarbons, such as undesirable methane and also deposition of coke, whereas lower temperatures generally should increase yields of ethylene, propylene, butylene and heavier molecular weight hydrocarbons. In the case of the alkyl bromide reaction over the 10-X zeolite catalyst, for example, it is believed that cyclization reactions also may occur such that the C7+ fractions contain substantial substituted aromatics. At increasing temperatures approaching about 400° C., for example, it is believed that brominated methane conversion generally should increase towards about 90% or greater; however, selectivity towards C₅+ hydrocarbons generally should decrease with increased selectivity toward lighter products, such as olefins. At temperatures exceeding about 550° C., for example, it is believed that a high conversion of brominated methane to methane and carbonaceous coke occurs. In the temperature range of between about 300° C. and about 450° C., as a byproduct of the reaction, a lesser amount of coke probably will build up on the catalyst over time during operation, causing a decline in catalyst activity over a range of hours, up to hundreds of hours, depending on the reaction conditions and the composition of the feed gas. Conversely, temperatures at the lower end of the range (e.g., below about 300° C.), may also contribute to coking due to a reduced rate of desorption of heavier products from the catalyst. Hence, operating temperatures within the range of about 250° C. to about 500° C., but preferably in the range of about 350° C. to about 450° C. in synthesis reactor 18 should generally balance increased selectivity of the desired olefins and C₅+ hydrocarbons and lower rates of deactivation due to carbon formation, against higher conversion per pass, which should minimize the quantity of catalyst, recycle rates and equipment size required.

Where, for example, the product hydrocarbons desired are primarily C3, C4, and C5+ gasoline-range and heavier hydrocarbon fractions, synthesis reactor 18 may be operated at a temperature within the range of about 150° C. to about 450° C. Temperatures above about 300° C. in synthesis reactor 18 may result in increased yields of light hydrocarbons, whereas lower temperatures generally may increase yields of heavier molecular weight hydrocarbons. By way of example, at the low end of the temperature range with brominated methane reacting over the ZSM-5 zeolite catalyst at temperatures as low as about 150° C., significant brominated methane conversion on the order of about 20% may occur, with a high selectivity towards C₅+ hydrocarbons. In the case of the brominated methane reaction over the ZSM-5 zeolite catalyst, for example, cyclization reactions also occur such that the C7+ fractions may be primarily comprise substituted aromatics. At increasing temperatures approaching about 300° C., for example, brominated methane conversion generally should increase towards about 90% or greater; however, selectivity towards C₅+ hydrocarbons generally may decrease and selectivity towards lighter products, particularly undesirable methane, may increase. Surprisingly, benzene, ethane or C₂,-C₃ olefin components are not typically present, or present in only very small quantities, in the reaction effluent, in accordance with certain embodiments, such as when a ZSM-5 catalyst is used at temperatures of about 390° C. However, at temperatures approaching about 45020 C., for example, almost complete conversion of brominated methane to methane and carbonaceous coke may occur. In the operating temperature range of between about 350° C. and about 420° C., as a byproduct of the reaction, a small amount of carbon may build up on the catalyst over time during operation, potentially causing a decline in catalyst activity over a range of hours, up to several days, depending on the reaction conditions and the composition of the feed gas. It is believed that higher reaction temperatures (e.g., above about 420° C.), associated with the formation of methane, favor the thermal cracking of brominated alkanes and formation of carbon or coke and hence an increase in the rate of deactivation of the catalyst. Conversely, temperatures at the lower end of the range (e.g., below about 350° C.) may also contribute to coking due to a reduced rate of desorption of heavier products from the catalyst. Hence, operating temperatures within the range of about 150° C. to about 450° C., but preferably in the range of about 350° C. to about 420° C., and most preferably, in the range of about 370° C. to about 400° C., in synthesis reactor 18 should generally balance increased selectivity of the desired C₅+ hydrocarbons and lower rates of deactivation due to carbon formation, against higher conversion per pass, which minimizes the quantity of catalyst, recycle rates and equipment size required.

The catalyst may be periodically regenerated in situ, by isolating synthesis reactor 18 from the normal process flow and purging with an inert gas, for example, at a pressure in a range of about 1 atm to about 5 atm at an elevated temperature in the range of about 400° C. to about 650° C. to remove unreacted material adsorbed on the catalyst insofar as is practical. Then, the deposited coke may be oxidized to CO₂, CO, and H₂O by addition of air or inert gas-diluted oxygen to synthesis reactor 18, for example, at a pressure in the range of about 1 atm to about 5 atm at an elevated temperature in the range of about 400° C. to about 650° C. The oxidation products and residual air or inert gas may be vented from synthesis reactor 18 during the regeneration period. However, as the regeneration off-gas may contain small amounts of bromine-containing species, as well as excess unreacted oxygen, the regeneration gas effluent may be directed into the oxidation portion of the process, wherein the bromine-containing species may be converted to elemental bromine and recovered for re-use within the process.

As illustrated in FIG. 3, synthesis outlet stream 20 may be withdrawn from synthesis reactor 18. In general, synthesis outlet stream 20 may comprise the product hydrocarbons and the additional hydrogen bromide generated in synthesis reactor 18. Synthesis outlet stream 20 further may comprise the hydrogen bromide generated in bromination reactor 10 and possibly unreacted alkanes. By way of example, synthesis outlet stream 20 may comprise olefins, C5+ hydrocarbons, and the additional hydrogen bromide. By way of further example, the synthesis outlet stream 20 may comprise C3, C4 and C5+ gasoline-range and heavier hydrocarbon fractions, as well as the additional hydrogen bromide. In certain embodiments, the hydrocarbons present in the synthesis outlet stream 20 may primarily comprise aromatics. In certain embodiments, the C7+ fraction of the hydrocarbons present in synthesis outlet stream 20 may primarily comprise substituted aromatics.

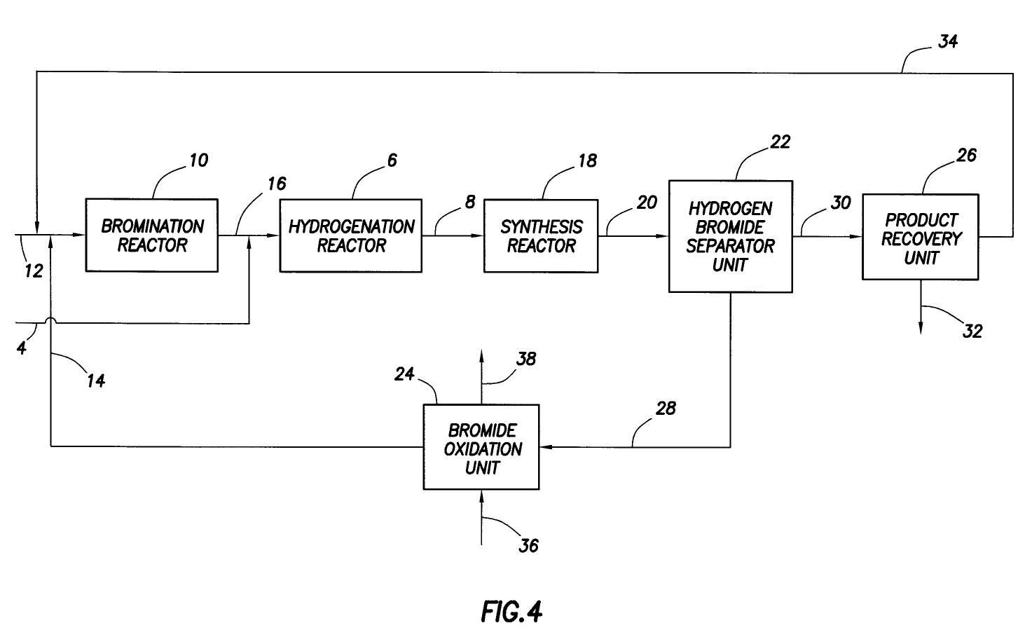

Referring to FIG. 4, an example block diagram of the process for the production of product hydrocarbons of FIG. 3 is illustrated that further includes product recovery and a wet process for bromine recovery and recycle, in accordance with one embodiment of the present invention. In the illustrated embodiment, the process includes bromination reactor 10, hydrogenation reactor 6, synthesis reactor 18, hydrogen bromide separator unit 22, bromide oxidation unit 24, and product recovery unit 26. Examples of processes that include bromination, synthesis, bromine recovery and recycle, and product recovery are described in more detail in U.S. Pat. No. 7,244,867, U.S. Pat. No. 7,348,464, and U.S. Patent Pub. No. 2006/0100469, the entire disclosures of which incorporated herein by reference.

As illustrated in FIG. 4, a gaseous feed stream 12 comprising alkanes may be combined with bromine stream 14 and the resulting mixture may be introduced into bromination reactor 10. In bromination reactor 10, the alkanes may be reacted with the bromine to form brominated alkanes and hydrogen bromide. Brominated stream 16 may be withdrawn from bromination reactor 10. In general, brominated stream 16 withdrawn from bromination reactor 10 comprises brominated alkanes, which may comprise multi-brominated alkanes, and hydrogen bromide. In the illustrated embodiment, brominated stream 16 may be combined with hydrogen stream 4 and introduced into hydrogenation reactor 6. In hydrogenation reactor 6, the multi-brominated alkanes present in brominated stream 16 may react with the hydrogen to form hydrogen bromide and one or more brominated alkanes with fewer bromine substituents. A hydrogenated stream 8 comprising the hydrogen bromide and the brominated alkane with fewer bromine substituents may be withdrawn from hydrogenation reactor 6 and introduced into synthesis reactor 18. Hydrogenated stream 8 also comprises mono-brominated alkanes that were produced in bromination reactor 10. In synthesis reactor 18, the brominated alkanes may be reacted exothermically in the presence of a catalyst to form product hydrocarbons and additional hydrogen bromide. Synthesis outlet stream 20 may be withdrawn from synthesis reactor 18. In general, synthesis outlet stream 20 may comprise the product hydrocarbons and the additional hydrogen bromide generated in synthesis reactor 18. Synthesis outlet stream 20 further may comprise the hydrogen bromide generated in bromination reactor 10 and possibly unreacted alkanes.

As set forth above, the process of FIG. 4 further includes hydrogen bromide separator unit 22. In the illustrated embodiment, synthesis outlet stream 20 may be introduced to hydrogen bromide separator unit 22. In hydrogen bromide separator unit 22, at least a portion of the hydrogen bromide present in synthesis outlet stream 20 may be separated from the product hydrocarbons. In certain embodiments, greater than about 98% and up to nearly 100% of the hydrogen bromide may be separated from the product hydrocarbons. An example of a suitable process for use in hydrogen bromide separator unit 22 may include contacting synthesis outlet stream 20, which may be a gas, with a liquid. Hydrogen bromide present in synthesis outlet stream 20 may be dissolved in the liquid and the mixture may be removed from hydrogen bromide separator unit 22 via hydrogen bromide stream 28. As described in more detail below, hydrocarbon stream 30 that may comprise the product hydrocarbons may be removed from hydrogen bromide separator unit 22.

One example of a suitable liquid that may be used to scrub the hydrogen bromide from the product hydrocarbons includes water. In these embodiments, the hydrogen bromide dissolves into the water and is at least partially ionized, forming an aqueous acid solution. Another example of a suitable liquid that may be used to scrub the hydrogen bromide from the product hydrocarbons includes an aqueous partially oxidized metal bromide salt solution containing metal hydroxide species, metal oxy-bromide species, metal oxide species, or mixtures thereof. The hydrogen bromide dissolved in the partially oxidized metal bromide salt solution should be neutralized by the metal hydroxide species, metal oxy-bromide species, metal oxide species, or mixtures thereof to form metal bromide salt in the hydrogen bromide stream 28 that may be removed from hydrogen bromide separator unit 22. Examples of suitable metals of the bromide salt include Fe(III), Cu(II), and Zn(II), as these metals may be less expensive and may be oxidized at lower temperatures, for example, in the range of about 120° C. to about 200° C. However, other metals that form oxidizable bromide salts may also be used. In certain embodiments, alkaline earth metals which may also form oxidizable bromide salts, such as Ca(II) or Mg(II) may be used.

As previously mentioned, the process further may include bromide oxidation unit 24. In the illustrated embodiment, hydrogen bromide stream 28 may be removed from hydrogen bromide separator unit 22 and introduced to bromide oxidation unit 24. In general, hydrogen bromide stream 28 may comprise water with one or more of a hydrogen bromide or a metal bromide salt dissolved therein. In bromide oxidation unit 24, the bromide salt present in the hydrogen bromide stream 28 may be oxidized to form elemental bromine, water, and the original metal hydroxide or metal oxy-bromide species (or metal oxides in the embodiment of a supported metal bromide salt). Oxygen stream 36 may be used to supply the oxygen needed for the oxidation to bromide oxidation unit 24. Oxygen stream 36 may comprise oxygen, air, or another suitable source of oxygen. Water stream 38 comprising the water formed in bromide oxidation unit 24 may be removed from bromide oxidation unit 24. While not illustrated, in certain embodiments, water stream 38 may be recycled to hydrogen bromide separator unit 22 as the liquid used for scrubbing the hydrogen bromide from the product hydrocarbons.

Oxidation in bromide oxidation unit 24 may occur, for example, at a temperature, of about 100° C. to about 600° C. and, alternatively, of about 120° C. to about 180° C. and a pressure of about ambient to about 5 atm. If the hydrogen bromide has not been neutralized prior to bromide oxidation unit 24 the hydrogen bromide may be neutralized in bromide oxidation unit 24 to form the bromide salt. By way of example, the hydrogen bromide may be neutralized with a metal oxide to form a metal bromide salt. Examples of suitable metals salts include Cu(II), Fe(III), and Zn(II), although other transition metals that form oxidizable bromide salts may also be used. In certain embodiments, alkaline earth metals which may also form oxidizable bromide salts, such as Ca(II) or Mg(II) may be used.

As illustrated in FIG. 4, bromine stream 14 may be removed from bromide oxidation unit 24. Bromine stream 14 generally may comprise the elemental bromine formed in bromide oxidation unit 24. In certain embodiments, bromine stream 14 may be removed from bromide oxidation unit 24 as a liquid. In the illustrated embodiment, bromine stream 14 may be recycled and combined with gaseous feed stream 12, as described above. Accordingly, the bromine may be recovered and recycled within the process.

As noted above, hydrocarbon stream 30 comprising the product hydrocarbons may be removed from hydrogen bromide separator unit 22. In general, hydrocarbon stream 30 comprises the product hydrocarbons from which the hydrogen bromide was separated. As illustrated in FIG. 4, hydrocarbon stream 30 may be introduced to product recovery unit 26 to recover, for example, the C5+ hydrocarbons as liquid product stream 32. Liquid product stream 32 may comprise, for example, C5+ hydrocarbons, including branched alkanes and substituted aromatics. In certain embodiments, liquid product stream 32 may comprise olefins, such as ethylene, propylene, and the like. In certain embodiments, the liquid product may comprise various hydrocarbons in the liquefied petroleum gas and gasoline-fuels range, which may include a substantial aromatic content, significantly increasing the octane value of the hydrocarbons in the gasoline-fuels range. While not illustrated, in certain embodiments, product recovery unit 26 may include dehydration and liquids recovery. Any conventional method of dehydration and liquids recovery, such as solid-bed dessicant adsorption followed by refrigerated condensation, cryogenic expansion, or circulating absorption oil or other solvent, as used to process natural gas or refinery gas streams, and to recover product hydrocarbons, may be employed in embodiments of the present invention.

At least a portion of the residual vapor effluent from product recovery unit 26 may be recovered as alkane recycle stream 34. Alkane recycle stream 34 may comprise, for example, methane and potentially other unreacted lower molecular weight alkanes. As illustrated, alkane recycle stream 34 may be recycled and combined with gaseous feed stream 12. In certain embodiments, alkane recycle stream 34 that is recycled may be at least 1.5 times the feed gas molar volume. While not illustrated in FIG. 4, in certain embodiments, another portion of the residual vapor effluent from product recovery unit 26 may be used as fuel for the process. Additionally, while also not illustrated in FIG. 4, in certain embodiments, another portion of the residual vapor effluent from product recovery unit 26 may be recycled and used to dilute the brominated alkane concentration introduced into synthesis reactor 18. Where used to dilute the brominated alkane concentration, the residual vapor effluent generally should be recycled at a rate to absorb the heat of reaction such that synthesis reactor 18 is maintained at the selected operating temperature, for example, in the range of about 300° C. to about 450° C. in order to maximize conversion versus selectivity and to minimize the rate of catalyst deactivation due to the deposition of carbonaceous coke. Thus, the dilution provided by the recycled vapor effluent should permit selectivity of bromination in bromination reactor 10 to be controlled in addition to moderating the temperature in synthesis reactor 18.

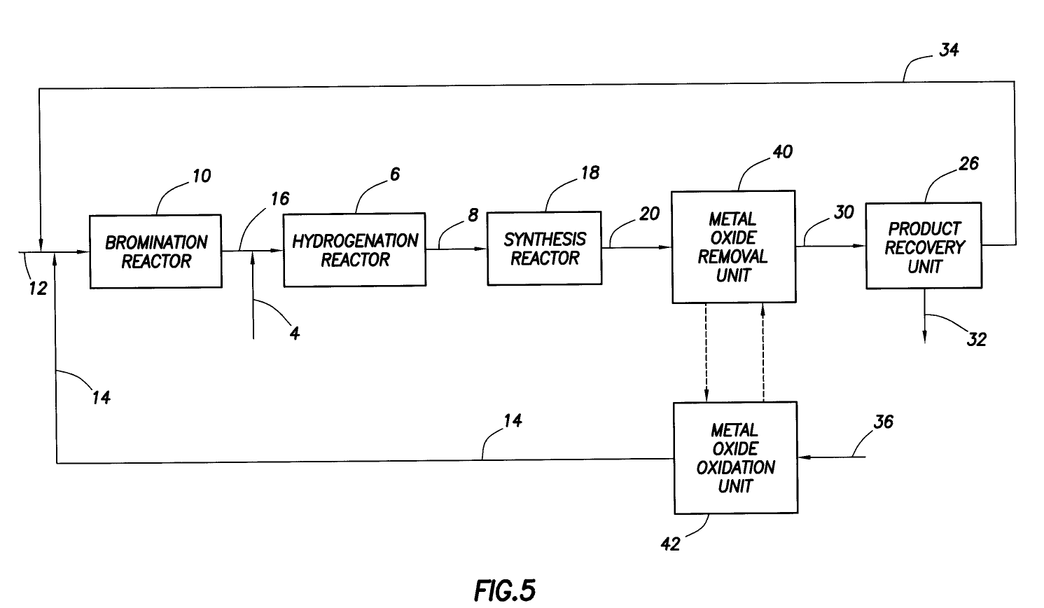

Referring to FIG. 5, an example block diagram of the process for the production of product hydrocarbons of FIG. 3 is illustrated that further includes product recovery and a dry process for bromine recovery and recycle, in accordance with one embodiment of the present invention. In the illustrated embodiment, the process includes bromination reactor 10, hydrogenation reactor 6, synthesis reactor 18, metal oxide HBr removal unit 40, metal bromide oxidation unit 42, and product recovery unit 26.

As illustrated in FIG. 5, a gaseous feed stream 12 comprising alkanes may be combined with bromine stream 14 and the resulting mixture may be introduced into bromination reactor 10. In bromination reactor 10, the alkanes may be reacted with the bromine to form brominated alkanes and hydrogen bromide. Brominated stream 16 may be withdrawn from bromination reactor 10. In general, brominated stream 16 withdrawn from bromination reactor 10 comprises brominated alkanes, which may comprise multi-brominated alkanes, and hydrogen bromide. In the illustrated embodiment, brominated stream 16 may be combined with hydrogen stream 4 and introduced into hydrogenation reactor 6. In hydrogenation reactor 6, the multi-brominated alkanes present in brominated stream 16 may react with the hydrogen to form hydrogen bromide and brominated alkanes with fewer bromine substituents. Hydrogenated stream 8 comprising the hydrogen bromide and the brominated alkane with fewer bromine substituents may be withdrawn from hydrogenation reactor 6 and introduced into synthesis reactor 18. Hydrogenated stream 8 also may comprise mono-brominated alkanes that were produced in bromination reactor 10. In synthesis reactor 18, the brominated alkanes may be reacted exothermically in the presence of a catalyst to form product hydrocarbons and additional hydrogen bromide. Synthesis outlet stream 20 may be withdrawn from synthesis reactor 18. In general, synthesis outlet stream 20 may comprise the product hydrocarbons and the additional hydrogen bromide generated in synthesis reactor 18. Synthesis outlet stream 20 further may comprise the hydrogen bromide generated in bromination reactor 10 and possibly unreacted alkanes.

As set forth above, the process of FIG. 5 further includes metal oxide HBr removal unit 40. In the illustrated embodiment, synthesis outlet stream 20 may be introduced to metal oxide HBr removal unit 40. An example of a suitable process for use in metal oxide HBr removal unit 40 may include reacting the hydrogen bromide present in synthesis outlet stream 20 with a metal oxide to form a bromide salt and steam. In the case of gaseous hydrogen bromide reacting with a metal oxide, such as magnesium oxide, the formation of the metal bromide salt and steam occurs in accordance with the following general reaction:

2HBr(g)+MgO→MgBr²+H²O(g) (3)

Accordingly, the hydrogen bromide may be separated from the product hydrocarbons. In certain embodiments, at least about 90% and potentially up to nearly 100% of the hydrogen bromide may be removed from the product hydrocarbons. As described in more detail below, hydrocarbon stream 30, that may comprise the product hydrocarbons, excess unreacted alkanes, and the steam, may be removed from metal oxide HBr removal unit 40.

The hydrogen bromide may be reacted with the metal oxide in metal oxide HBr removal unit 40, for example, at a temperature of less than about 600° C. and, alternatively, of between about 50° C. to about 500° C. By way of example, metal oxide HBr removal unit 40 may include a vessel or reactor that contains a bed of solid-phase metal oxide. In certain embodiments, reaction of the hydrogen bromide with the solid-phase metal oxide forms steam and a solid phase metal bromide. Examples of suitable metals for the metal oxide include, but are not limited to, magnesium (Mg), calcium (Ca), vanadium (V), chromium (Cr), manganese (Mn), iron (Fe), cobalt (Co), nickel (Ni), copper (Cu), zinc (Zn), or tin (Sn). Magnesium, copper, or iron, wherein the reaction with the hydrogen bromide to form the bromide salt may be reversible at a temperature of less than about 500° C. may be used, in certain embodiments. However, it should be noted that with certain metal oxides, for example copper and iron, the reaction temperature with hydrogen bromide should be limited to less than about 200° C. and 100° C., respectively, to substantially avoid the thermal decomposition of the metal bromide to the reduced metal bromide salt and elemental bromine which could result in undesirable bromination of the hydrocarbon products. With certain metal oxides, for example nickel oxide, it may also be important to limit the temperature of the metal oxide reaction with the hydrogen bromide to substantially avoid the possibility of oxidation of the hydrocarbons by the metal oxide. In certain embodiments, the solid metal oxide may be immobilized on a suitable attrition-resistant support, for example, silica or alumina, etc. It has been found that inert supports with low to medium specific surface area, preferably in the range of about 1 to 400 m2/g, and more preferably in the range of about 5 to 50 m2/g, are advantageous in minimizing the adsorption of hydrocarbons, while still allowing sufficient area for relatively high loading of metal oxide with good dispersion to effect a high capacity for hydrogen bromide removal, in certain embodiments of the present invention.

As previously mentioned, the process further may include metal bromide oxidation unit 42. In accordance with certain embodiments of the present invention, the metal bromide oxidation unit 42 may include contacting the metal salt formed in the metal oxide HBr removal unit 40 with oxygen stream 36 to form the original metal oxide and elemental bromine. Oxygen stream 36 may comprise oxygen, air, or another suitable source of oxygen. In the case of the oxidation of the metal bromide salt, oxygen reacts with the metal bromide salt, such as magnesium bromide, in accordance with the following general reaction:

$${MgBr}^{2} + {2}^{+} {O}_{2} -> MgO + {Br}^{2} \qquad ( 4 )$$

In certain embodiments, the solid phase metal bromide may be contacted with a gas comprising oxygen, for example, at a temperature of about 100° C. to about 500° C. As will be appreciated by those of ordinary skill in the art, with the benefit of this disclosure, the dry process may include at least two vessels or reactors operating in a cyclic fashion, in certain embodiments. By way of example, one of the vessels or reactors may be used as metal oxide HBr removal unit 40 for removing the hydrogen bromide via reaction with metal oxide while the other reactor or vessel is used as metal bromide oxidation unit 42 for oxidizing the metal bromide to form elemental bromine.

As illustrated in FIG. 5, bromine stream 14 may be removed from metal bromide oxidation unit 42. Bromine stream 14 generally may comprise the elemental bromine formed in metal bromide oxidation unit 42. In certain embodiments, bromine stream 14 may be removed from metal bromide oxidation unit 42 as a liquid. In the illustrated embodiment, bromine stream 14 may be recycled and combined with gaseous feed stream 12, as described above. Accordingly, the bromine may be recovered and recycled within the process.

As noted above, hydrocarbon stream 30 comprising the product hydrocarbons may be removed from metal oxide HBr removal unit 40. In general, hydrocarbon stream 30 comprises the products hydrocarbons and excess unreacted alkanes from which the hydrogen bromide was separated. As illustrated in FIG. 5, hydrocarbon stream 30 may be introduced to product recovery unit 26 to recover, for example, the C5+ hydrocarbons as liquid product stream 32. Liquid product stream 32 may comprise, for example, C5+ hydrocarbons, including branched alkanes and substituted aromatics. In certain embodiments, liquid product stream 32 may comprise olefins, such as ethylene, propylene, and the like. In certain embodiments, liquid product stream 32 may comprise various hydrocarbons in the liquefied petroleum gas and gasoline-fuels and heavier range, which may include a substantial aromatic content in the gasoline range, significantly increasing the octane value of the hydrocarbons in the gasoline-fuels range. While not illustrated, in certain embodiments, product recovery unit 26 may include dehydration and liquids recovery. Any conventional method of dehydration and liquids recovery, such as solid-bed dessicant adsorption followed by refrigerated condensation, cryogenic expansion, or circulating absorption oil or other solvent, as used to process natural gas or refinery gas streams, and to recover product hydrocarbons, may be employed in embodiments of the present invention.

At least a portion of the residual vapor effluent from product recovery unit 26 may be recovered as alkane recycle stream 34. Alkane recycle stream 34 may comprise, for example, methane and potentially other unreacted lower molecular weight alkanes. As illustrated, alkane recycle stream 34 may be recycled and combined with gaseous feed stream 12. In certain embodiments, alkane recycle stream 34 that is recycled may be at least 1.5 times the feed gas molar volume. While not illustrated in FIG. 5, in certain embodiments, another portion of the residual vapor effluent from product recovery unit 26 may be used as fuel for the process. Additionally, while also not illustrated in FIG. 5, in certain embodiments, another portion of the residual vapor effluent from product recovery unit 26 may be recycled and used to dilute the brominated alkane concentration introduced into synthesis reactor 18. Where used to dilute the brominated alkane concentration, the residual vapor effluent generally should be recycled at a rate to absorb the heat of reaction such that synthesis reactor 18 is maintained at the selected operating temperature, for example, in the range of about 150° C. to about 500° C. in order to maximize conversion versus selectivity and to minimize the rate of catalyst deactivation due to the deposition of carbonaceous coke. Thus, the dilution provided by the recycled vapor effluent should permit selectivity of bromination in bromination reactor 10 to be controlled in addition to moderating the temperature in synthesis reactor 18.

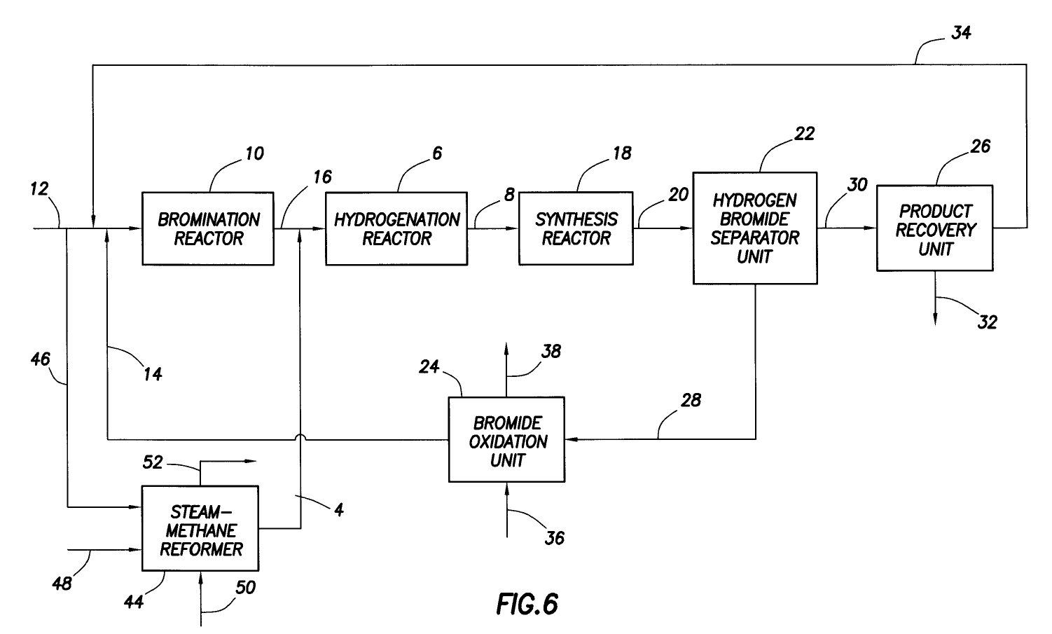

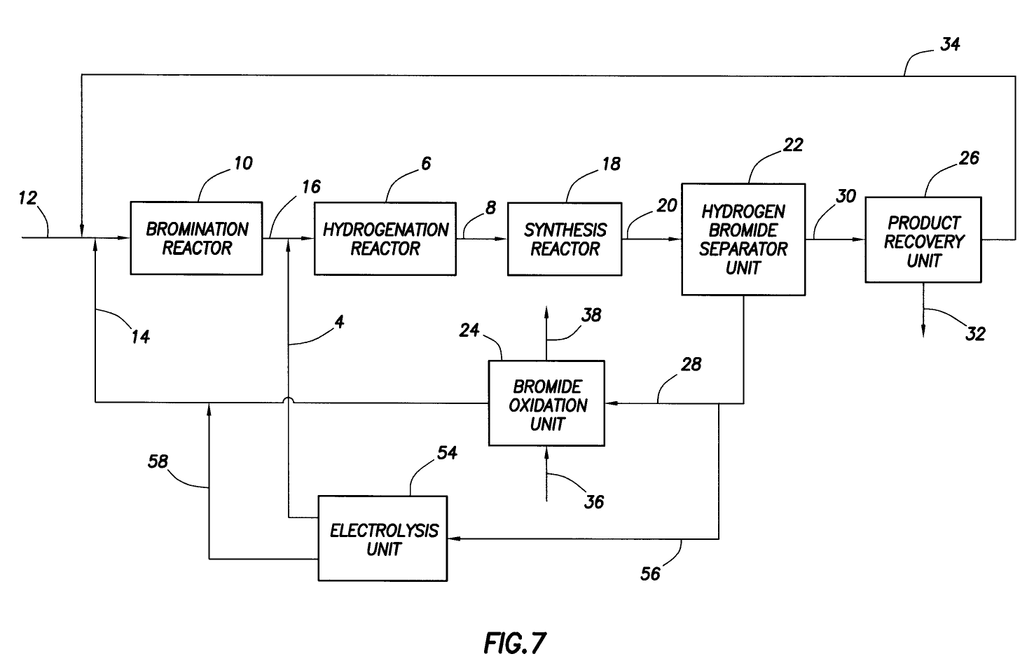

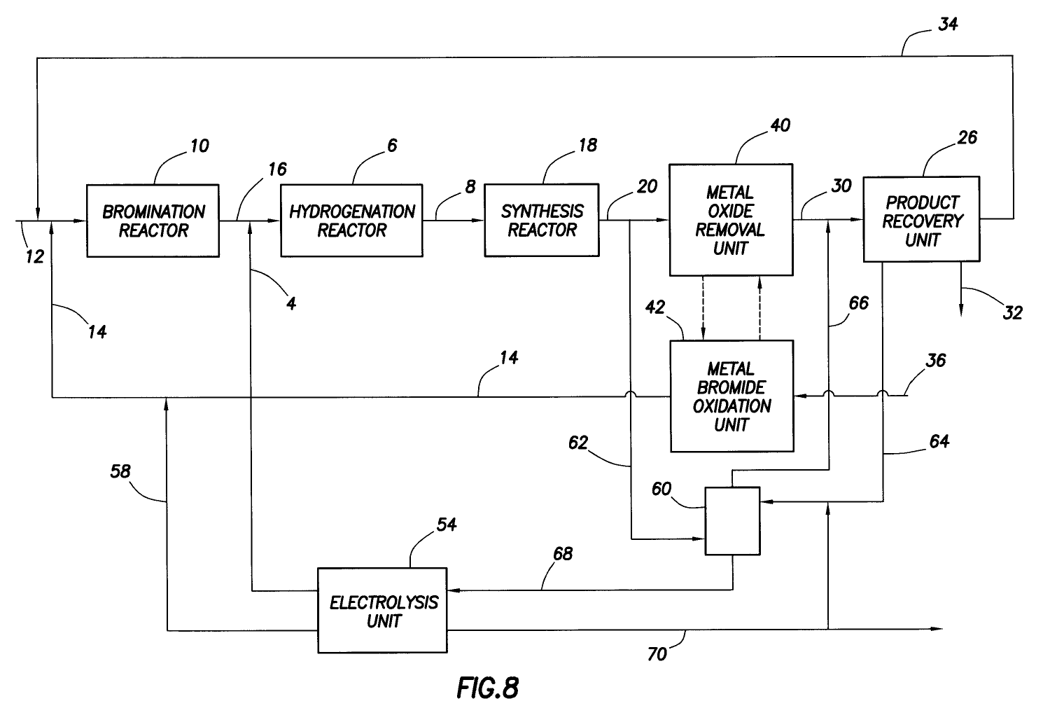

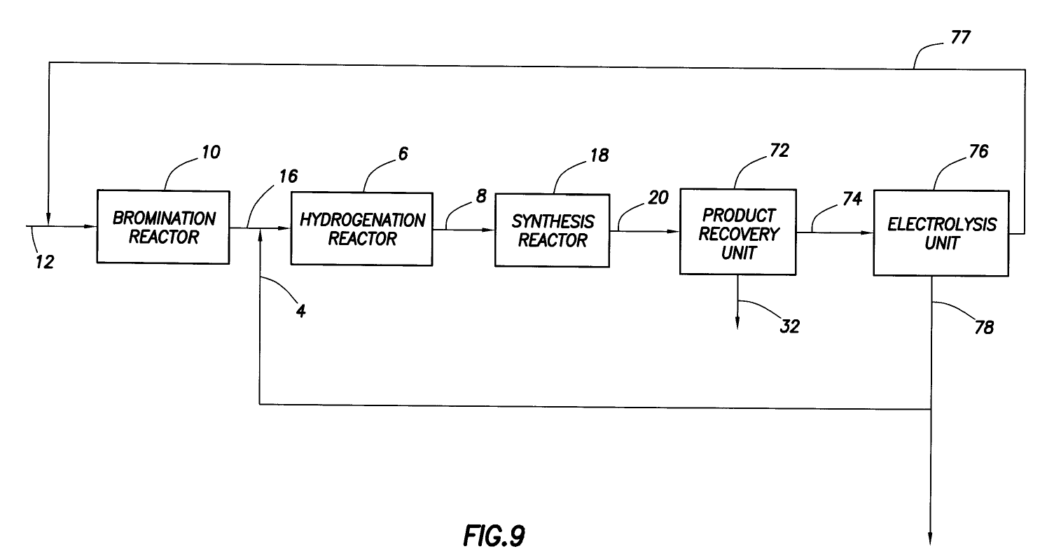

As described above with respect to FIG. 1, the hydrogen present in hydrogen stream 4 supplied to hydrogenation reactor 6 may be provided via any suitable source, including steam-methane reforming (“SMR”), the water-gas shift reaction of carbon monoxide, or electrolysis of water, metal halide salt, or hydrogen bromide. FIGS. 6-9 illustrated different embodiments of the present invention for providing hydrogen to hydrogenation reactor 6. FIG. 6 illustrates an embodiment of the present invention wherein steam-methane reforming is used to provide hydrogen for use in hydrogenation reactor 6. FIGS. 7-9 illustrate embodiments of the present invention wherein electrolysis used to provide hydrogen for use in hydrogenation reactor 6. FIGS. 7 and 8 illustrate embodiments of the present invention that include aqueous electrolysis, while FIG. 9 illustrates vapor-phase electrolysis.

Referring to FIG. 6, an example block diagram of the process for the production of product hydrocarbons of FIG. 4 is illustrated that further includes SMR, in accordance with one embodiment of the present invention. While FIG. 6 illustrates embodiments of FIG. 4 that include wet bromine recovery and recycle, those of ordinary skill in the art, with the benefit of this disclosure, will appreciate that the embodiments of FIG. 5 that include dry bromine recovery and recycle with SMR may also be used in accordance with embodiments of the present invention. In the illustrated embodiment, steam-methane reformer 44 is used to provide hydrogen for use in hydrogenation reactor 6. As illustrated, SMR feed stream 46 may be supplied to steam-methane reformer 44. In general, SMR feed stream 46 may comprise a portion of gaseous feed stream 12. Accordingly, SMR feed stream 46 may comprise, for example, lower molecular weight alkanes, whether naturally occurring or synthetically produced. Examples of suitable sources of lower molecular weight alkanes include, but are not limited to, natural gas, coalbed methane, regasified liquefied natural gas, gas derived from gas hydrates, chlathrates or combinations thereof, gas derived from anaerobic decomposition of organic matter or biomass, synthetically produced natural gas or alkanes, and combinations thereof. In certain embodiments, a sufficient amount of gaseous feed stream 12 may supplied to steam-methane reformer 44 via SMR feed stream 46 to provide at least about 1 mole of hydrogen per mole of the multi-brominated alkanes supplied to hydrogenation reactor 6 and, in certain embodiments, to provide at least one mole of hydrogen per mole of di-brominated methane.

In steam-methane reformer 44, the lower molecular weight hydrocarbons in SMR feed stream 46 may be reacted with steam in the presence of a catalyst, such as a nickel-based catalyst, for example. Steam may be supplied to steam-methane reformer 44 via water feed stream 48. In the illustrated embodiment, air feed 50 may provide oxygen to, for example, combust a portion of the gas feed and/or SMR process gas to provide the heat required for the endothermic reforming reactions. Steam-methane reformer 44 may operate, for example, at temperature of about 700° C. to about 1,100° C. In the case of methane, steam may react with methane in accordance with the following general reactions:

CH⁴(g)+H²O(g)→CO(g)+3H²(g) (5)

CO(g)+H²O(g)→CO²(g)+3H²(g) (6)

Hydrogen stream 4 comprising the hydrogen produced in steam-methane reformer 44 may be removed from steam-methane reformer 44 and supplied to hydrogenation reactor 6. As set forth above, the hydrogen may react in hydrogenation reactor 6 with multi-brominated alkanes to form hydrogen bromide and one or more brominated alkanes with fewer bromine substituents. In addition to hydrogen stream 4, carbon dioxide/water stream 52 comprising carbon dioxide and water may also be removed from steam-methane reformer 44.

Referring to FIG. 7 an example block diagram of the process for the production of product hydrocarbons of FIG. 4 is illustrated that further includes electrolysis, in accordance with one embodiment of the present invention. In the illustrated embodiment, liquid-phase electrolysis unit 54 is used to provide hydrogen for use in hydrogenation reactor 6. As illustrated, hydrogen bromide feed stream 56 may be supplied to electrolysis unit 54. In general, hydrogen bromide feed stream 56 may comprise a portion of hydrogen bromide stream 28 that may be removed from hydrogen bromide separator unit 22. Accordingly, hydrogen bromide feed stream 56 may comprise, for example, water and hydrogen bromide dissolved in the water.

In liquid-phase electrolysis unit 54, bromine may be recovered from the hydrogen bromide present in hydrogen bromide feed stream 56. Electric energy may be used to electrolyze at least a portion of the hydrogen bromide to form elemental bromine and hydrogen. In the electrolysis of an aqueous hydrochloric acid solution (HCl), the Uhde process may be used and may also possibly be adapted for the electrolysis of the aqueous hydrobromic acid, e.g., the hydrogen bromide dissolved in hydrogen bromide feed stream 56. While not illustrated in FIG. 9, one or more electrolysis cells may be included in liquid-phase electrolysis unit 54. Those of ordinary skill in the art, with the benefit of this disclosure, will appreciate that the electrolysis cells may be operated in parallel or series, in accordance with certain embodiments of the present invention. In the electrolysis of the hydrogen bromide, electric energy may be passed through hydrogen bromide feed stream 56 that comprises water and hydrogen bromide dissolved therein with the production of bromine at the anode and hydrogen at the cathode of the electrolysis cells. While not illustrated, the energy required to separate the hydrogen and the bromine may be provided by an electrical power supply.

By way of example, the electrolysis of hydrogen bromide may occur in accordance with the following general half-reactions occurring at the anode and cathode electrodes, respectively, of the electrolysis cells:

2Br(−)→Br₂+2e³¹ (7)

2H(+)+2e⁻→H₂ (8)

In certain embodiments, a sufficient amount of hydrogen bromide stream 28 may be supplied to electrolysis unit 54 via hydrogen bromide feed stream 56 to provide at least about 1 mole of hydrogen per mole of the multi-brominated alkanes supplied to hydrogenation reactor 6 and, in certain embodiments, to provide at least one mole of hydrogen per mole of di-brominated methane.

Hydrogen stream 4 comprising the hydrogen produced in liquid-phase electrolysis unit 54 may be removed therefrom and supplied to hydrogenation reactor 6. As set forth above, the hydrogen may react in hydrogenation reactor 6 with multi-brominated alkanes to form hydrogen bromide and one or more brominated alkanes with fewer bromine substituents. In addition to hydrogen stream 4, produced bromine stream 58 comprising the bromine produced in liquid-phase electrolysis unit electrolysis unit 54 may be removed and combined with bromine stream 14 that is supplied to bromination reactor 10.

In the case of an oxidized aqueous metal salt solution being used to scrub out the hydrogen bromide such that the hydrogen bromide would be neutralized to form the metal bromide salt and water, hydrogen bromide feed stream 56 to liquid-phase electrolysis unit 54 would comprise the metal bromide salt and water. In these embodiments, the aqueous metal bromide could be electrolyzed to produce elemental bromine and the reduced metal ion or elemental metal. By way of further example, the electrolysis of a metal bromide salt (e.g., Fe(III)Br₂) may occur in accordance with the following general half-reactions occurring at the anode and cathode electrodes, respectively, of the electrolysis cells:

2Br(−)→Br₂+2e⁻ (9)

2Fe(+3)+2e⁻→2Fe(+2) (10)

In certain embodiments, air or oxygen may be passed over the cathode to further oxidize the metal ion (e.g., the ferrous ion) to metal hydroxide and partially depolarize the electrode according to the following reaction:

1.333Fe(+2)+O²+2H²O+2.667e⁻→1.333Fe(OH)³ (11)

Referring to FIG. 8, an example block diagram of the process for the production of product hydrocarbons of FIG. 5 is illustrated that further includes electrolysis, in accordance with one embodiment of the present invention. In the illustrated embodiment, liquid-phase electrolysis unit 54 is used to provide hydrogen for use in hydrogenation reactor 6. As illustrated, the process further includes liquid-phase electrolysis unit 54 and hydrogen bromide absorber 60. A portion of synthesis outlet stream 20 may bypass around metal oxide HBr removal unit 40 and be supplied to hydrogen bromide absorber 60 via absorber feed stream 62. Accordingly, absorber feed stream 62 may comprise product hydrocarbons and hydrogen bromide. In certain embodiments, a sufficient amount of hydrogen bromide may be bypassed to provide at least about 1 mole of hydrogen per mole of the multi-brominated alkanes supplied to hydrogenation reactor 6 and, in certain embodiments, to provide at least one mole of hydrogen per mole of di-brominated methane.

In hydrogen bromide absorber 60, the hydrogen bromide may be separated from the product hydrocarbons present in absorber feed stream 62. An example of a suitable process for separating the hydrogen bromide from the product hydrocarbons includes contacting absorber feed stream 62, which may be a gas, with a liquid, such as scrubbing stream 64. Hydrogen bromide present in absorber feed stream 62 may be dissolved in the liquid. One example of a suitable liquid that may be used to scrub out the hydrogen bromide from the product hydrocarbons includes water. As illustrated, scrubbing stream 64 may include water from product recovery unit 26. In these embodiments, the hydrogen bromide dissolves into the water and is at least partially ionized, forming an aqueous acid solution. In other embodiments, as described above, an oxidized aqueous metal salt solution may be used to scrub out the hydrogen bromide such that the hydrogen bromide would be neutralized to form a metal bromide salt and water. Scrubbed hydrocarbon stream 66 comprising the product hydrocarbons from which the hydrogen bromide has been scrubbed may then be provided to product recovery unit 26, and electrolysis feed stream 68 comprising water and hydrogen bromide (or metal bromide salt) dissolved therein may be provided to liquid-phase electrolysis unit 54.

In liquid-phase electrolysis unit 54, bromine may be recovered from the hydrogen bromide present in electrolysis feed stream 68. Electric energy may be used to electrolyze at least a portion of the hydrogen bromide to form elemental bromine and hydrogen. In the electrolysis of an aqueous hydrochloric acid solution (HCl), the Uhde process may be used and may also possibly be adapted for the electrolysis of the aqueous hydrobromic acid, e.g., the hydrogen bromide dissolved in electrolysis feed stream 68. In the electrolysis of the hydrogen bromide, electric energy may be passed through electrolysis feed stream 68 that comprises water and hydrogen bromide dissolved therein with the production of bromine at the anode and hydrogen at the cathode of the electrolysis cells. The electrolysis of hydrogen bromide may occur in accordance with the half-reactions set forth above in equations (7) and (8). In the case of an oxidized aqueous metal salt solution being used to scrub out the hydrogen bromide such that the hydrogen bromide would be neutralized to form the metal bromide salt and water, the aqueous metal bromide could be electrolyzed to produce elemental bromine and the reduced metal ion or elemental metal. The electrolysis of the metal bromide salt (e.g., Fe(III)Br₂) may occur in accordance with the half-reactions set forth above in equations (9) and (10). In certain embodiments, air or oxygen may be passed over the cathode to further oxidize the metal ion (e.g., the ferrous ion) to metal hydroxide and partially depolarize the electrode according to the reaction set forth above in equation (11).

In certain embodiments, a sufficient amount of hydrogen bromide stream 28 may be supplied to electrolysis unit 54 via hydrogen bromide feed stream 56 to provide at least about 1 mole of hydrogen per mole of the multi-brominated alkanes supplied to hydrogenation reactor 6 and, in certain embodiments, to provide at least one mole of hydrogen per mole of di-brominated methane.

Hydrogen stream 4 comprising the hydrogen produced in liquid-phase electrolysis unit 54 may be removed therefrom and supplied to hydrogenation reactor 6. As set forth above, the hydrogen may react in hydrogenation reactor 6 with multi-brominated alkanes to form hydrogen bromide and one or more brominated alkanes with fewer bromine substituents. In addition to hydrogen stream 4, produced bromine stream 58 comprising the bromine produced in liquid-phase electrolysis unit 54 may be removed and combined with bromine stream 14 that is supplied to bromination reactor 10.

Referring to FIG. 9 an example block diagram of the process for the production of product hydrocarbons of FIG. 3 is illustrated that further includes product recovery and electrolysis, in accordance with one embodiment of the present invention. As illustrated, the process further includes product recovery unit 72 and vapor-phase electrolysis unit 76. In the illustrated embodiment, vapor-phase electrolysis unit 76 is used for the gas-phase electrolysis of hydrogen bromide produced in the process to provide hydrogen for use in hydrogenation reactor 6. In addition, the embodiment of FIG. 9 also may produce excess hydrogen as a product.

As illustrated in FIG. 9, synthesis outlet stream 30 may be introduced to product recovery unit 72 to recover, for example, the C5+ hydrocarbons as liquid product stream 32. Liquid product stream 32 may comprise, for example, C5+ hydrocarbons, including branched alkanes and substituted aromatics. In certain embodiments, liquid product stream 32 may comprise olefins, such as ethylene, propylene, and the like. In certain embodiments, liquid product stream 32 may comprise various hydrocarbons in the liquefied petroleum gas and gasoline-fuels range, which may include a substantial aromatic content, significantly increasing the octane value of the hydrocarbons in the gasoline-fuels range. While not illustrated, in certain embodiments, product recovery unit 72 may include dehydration and liquids recovery. Any conventional method of dehydration and liquids recovery, such as solid-bed dessicant adsorption followed by refrigerated condensation, cryogenic expansion, or circulating absorption oil or other solvent, as used to process natural gas or refinery gas streams, and to recover product hydrocarbons, may be employed in embodiments of the present invention.

Vapor effluent stream 74 from product recovery unit 72 may be supplied to vapor-phase electrolysis unit 76. In certain embodiments, vapor effluent stream 74 may comprise methane and other unreacted lower molecular weight alkanes that were not recovered in product recovery unit 72. In addition, vapor effluent stream 74 further may comprise hydrogen bromide that was present in synthesis outlet stream 30 that was introduced to product recovery unit 72. In vapor-phase electrolysis unit 76, electrolysis of the hydrogen bromide may include using electric energy to electrolyze at least a portion of the hydrogen bromide to form elemental bromine at the anode and hydrogen at the cathode. The electrolysis of hydrogen bromide may occur in accordance with the half-reactions set forth above in equations (7) and (8). An example process for the vapor-phase electrolysis of hydrogen bromide is described in U.S. Pat. No. 5,411,641, the entire disclosure of which is incorporated herein by reference.

In one embodiment, vapor effluent stream 74 may be introduced through the inlet of an electrolysis cell comprising a cation-transporting membrane and an anode and a cathode each disposed in contact with a respective side of the membrane. In the electrolysis cell, molecules of the hydrogen bromide may be reduced at the anode to produce bromine gas and hydrogen cations. The hydrogen cations may be transported through the membrane to the cathode side where the protons hydrogen cations combine with electrons on the cathode to form hydrogen gas. Examples of suitable cation-transporting membranes include a cationic membrane that comprise fluoro or perfluoromonomers, such as a copolymer of two or more fluro or perfluoromonomers at least one of which contains pendant sulfonic acid groups. Another example of a suitable cation-transporting membrane includes proton-conducting ceramics, such as beta-alumina.

In another embodiment, vapor effluent stream 74 may be introduced to the cathode side of an electrolysis cell comprising an anion-transporting membrane (e.g., a molten-salt saturated membrane) with an anode and a cathode each disposed on opposite sides of the membrane. In the electrolysis cell, molecules of the hydrogen bromide may be reduced at the cathode, combining with electrons to produce hydrogen gas and bromide anions. The bromide anions may then be transported through the membrane to the anode side where the bromide anions liberate electrons and combine to form the bromine.

Product hydrogen stream 78 comprising the hydrogen produced in vapor-phase electrolysis unit 76 may be removed therefrom. A portion of product hydrogen stream 78 may be supplied to hydrogenation reactor 6 as hydrogen stream 4. In certain embodiments, a sufficient amount of hydrogen may be provided to hydrogenation reactor to provide at least about 1 mole of hydrogen per mole of the multi-brominated alkanes supplied to hydrogenation reactor 6 and, in certain embodiments, to provide at least one mole of hydrogen per mole of di-brominated methane. The remaining portion of hydrogen in product hydrogen stream 78 may be withdrawn from the process as a product. In certain embodiments, for example, where there may be no local need for hydrogen, two more electrolysis cells may be used in parallel, with one or more operated with an air-depolarized cathode in which is passed over the cathode, producing water vapor rather than hydrogen. Operating the cell with an air-depolarized cathode may reduce the voltage and power required for the electrolysis.

The bromine produced in vapor-phase electrolysis unit 76 may be recycled to bromination reactor 10 via alkane/bromine recycle stream 77. In addition to the bromine, alkane/bromine recycle stream 77 also may comprise at least a portion of the alkanes that were present in vapor effluent stream 74 that is introduced to vapor-phase electrolysis unit 76. Alkaneibromine recycle stream 77 may comprise, for example, bromine, methane, and potentially other unreacted lower molecular weight alkanes. As illustrated, alkane/recycle stream 34 may be recycled and combined with gaseous feed stream 12. The bromine in alkane/recycle stream 77 may react with gaseous feed stream 12 in bromination reactor 10. While not illustrated, in certain embodiments, gaseous feed stream may also be combined with make-up stream of bromine. In certain embodiments, the alkanes that are recycled in alkane/bromine recycle stream 34 may be at least 1.5 times the feed gas molar volume. While not illustrated in FIG. 9, in certain embodiments, another portion of the alkanes recovered from vapor-phase electrolysis unit 76 may be used as fuel for the process. Additionally, while also not illustrated in FIG. 9, in certain embodiments, another portion of the alkanes recovered from vapor-phase electrolysis unit 76 may be recycled and used to dilute the brominated alkane concentration introduced into synthesis reactor 18. Where used to dilute the brominated alkane concentration, the residual vapor effluent generally should be recycled at a rate to absorb the heat of reaction such that synthesis reactor 18 is maintained at the selected operating temperature, for example, in the range of about 300° C. to about 450° C. in order to maximize conversion versus selectivity and to minimize the rate of catalyst deactivation due to the deposition of carbonaceous coke. Thus, the dilution provided by the recycled vapor effluent should permit selectivity of bromination in bromination reactor 10 to be controlled in addition to moderating the temperature in synthesis reactor 18.

FIGS. 10-14 illustrate embodiments of the present invention for the production of product hydrocarbons wherein mono-brominated alkanes and hydrogen bromides are bypassed around hydrogenation reactor 6. Because hydrogenation reactor 6 in the configuration of FIGS. 10-14 has a reduced flow of more concentrated reactants than the series configuration previously described, hydrogenation reactor 6 may be of smaller size, potentially requiring less catalyst and reducing the pressure drop across the entire process.

As illustrated in FIGS. 10-14, brominated stream 16 may be removed from bromination reactor 10. In general, brominated stream 16 may comprise brominated alkanes and hydrogen bromide. The brominated alkanes present in brominated stream 16 may comprise mono-brominated alkanes and multi-brominated alkanes. For separation of the multi-brominated alkanes, brominated stream 16 may be introduced to first heat exchanger 80. Because the multi-brominated alkanes have a high boiling point related to other components of brominated stream 16, such as the mono-brominated alkanes, hydrogen bromide, and residual methane or other light alkanes, the multi-brominated alkanes may be readily condensed by cooling brominated stream 16 in first heat exchanger 80. Brominated stream 16 may be cooled, for example, to a temperature of about 10° C. to about 90° C.

Gaseous brominated effluent 82 may be removed from first heat exchanger 80 and reheated in second heat exchanger 84 to form synthesis reactor feed stream 86. In second heat exchanger 84, gaseous brominated effluent 82 may be heated, for example, to a temperature of about 300° C. to about 400° C. In general, gaseous brominated effluent 82 may comprise the portion of brominated stream 16 that was not condensed in first heat exchanger 80. By way of example, gaseous brominated effluent 82 may comprise mono-brominated alkanes, hydrogen bromide, residual methane or other light alkanes, and some residual multi-brominated alkanes that were not condensed.

Condensed brominated stream 88 may be removed from first heat exchanger 80 and vaporized in third heat exchanger 90 to form hydrogenation reactor feed stream 92. In third heat exchanger 90, condensed brominated stream 88 may be heated, for example, to a temperature of about 200° C. to about 450° C. to vaporize the multi-brominated alkanes. In general, condensed brominated stream 88 may comprise the portion of brominated stream 16 that was condensed in first heat exchange 80. By way of example, condensed brominated stream 88 may comprise multi-brominated alkanes and a small amount of mono-brominated alkanes that have condensed along with the multi-brominated alkanes. For example, at least of portion of the multi-brominated alkanes formed in bromination reactor 10 may be condensed in first heat exchanger 80 and then vaporized in third heat exchanger 90.

In the illustrated embodiment, hydrogenation reactor feed stream 92 from third heat exchanger 90 may be combined with hydrogen stream 4 and introduced into hydrogenation reactor 6. In hydrogenation reactor 6, the multi-brominated alkanes present in hydrogenation reactor feed stream 92 may react with the hydrogen to form hydrogen bromide and one or more brominated alkanes with fewer bromine substituents. In accordance with embodiments of the present invention, it is believed that hydrogenation reactor 6 may be operated to form mono-brominated alkanes and hydrogen bromide with a high, up to nearly 100% selectivity, in that essentially all the multi-brominated alkanes may be converted to mono-brominated alkanes. It is believed that higher temperature, while resulting in high apparent conversion of the multi-brominated alkanes, also accelerates coking. Thus, operation at lower temperatures, at the expense of requiring a larger reactor to achieve high conversion of the multi-brominated alkanes, may be acceptable due to the lower losses due to the formation of coke and slower catalyst deactivation. It has been found that high activity may be restored to the catalyst be regeneration with an oxygen-containing gas mixture or air.

Concentrated hydrogenated stream 94 comprising the hydrogen bromide and the brominated alkanes with fewer bromine substituents may be withdrawn from hydrogenation reactor 6. By way of example, concentrated hydrogenated stream 94 withdrawn from the hydrogenation reactor may comprise the hydrogen bromide and mono-brominated alkanes. Hydrogenation reactor 6 and hydrogen stream 4 are described in more detail with respect to FIG. 1 above. Concentrated hydrogenated stream 94 may be combined with synthesis reactor feed stream 86 and introduced to synthesis reactor 18. Synthesis reactor 18 and other components of FIGS. 10-14 are described in more detail with respect to the figures above.

To facilitate a better understanding of the present invention, the following examples of certain aspects of some embodiments are given. In no way should the following examples be read to limit, or define, the entire scope of the invention.

### EXAMPLE 1

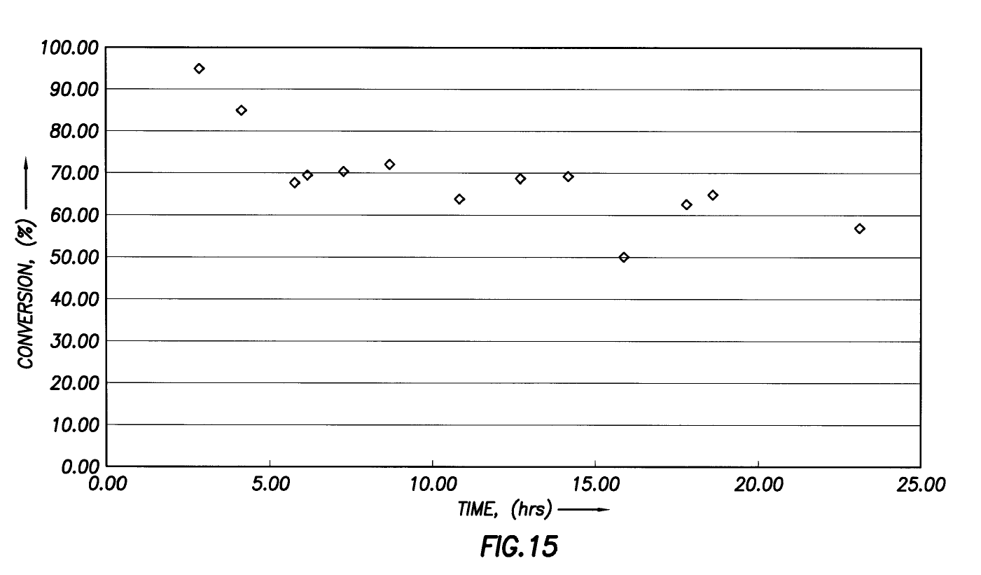

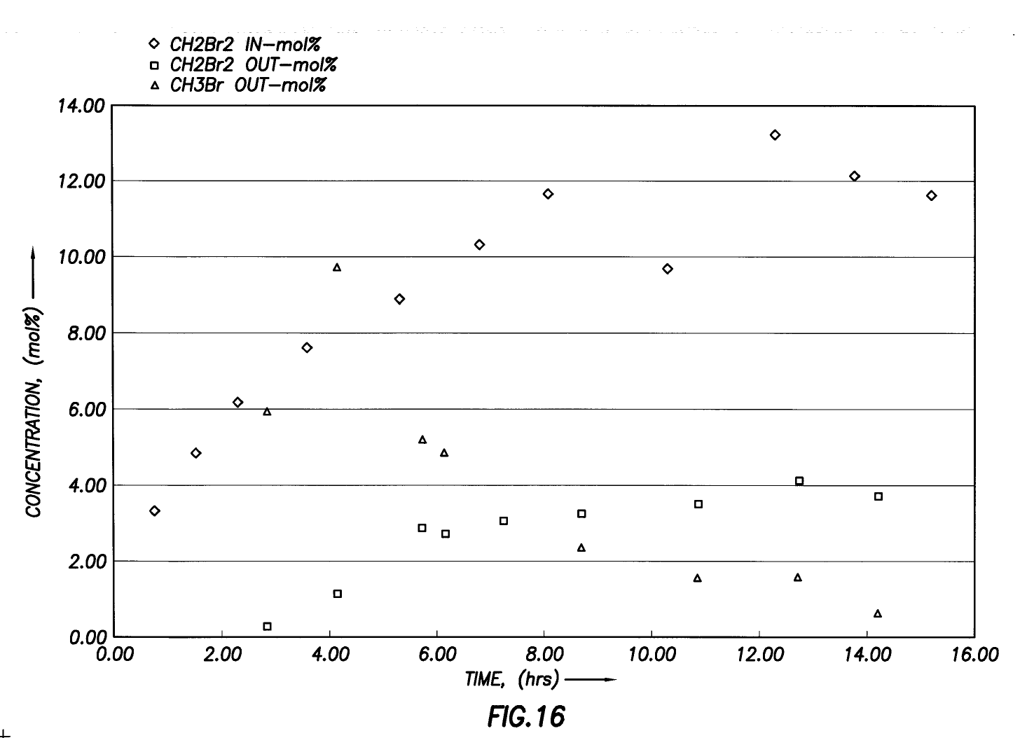

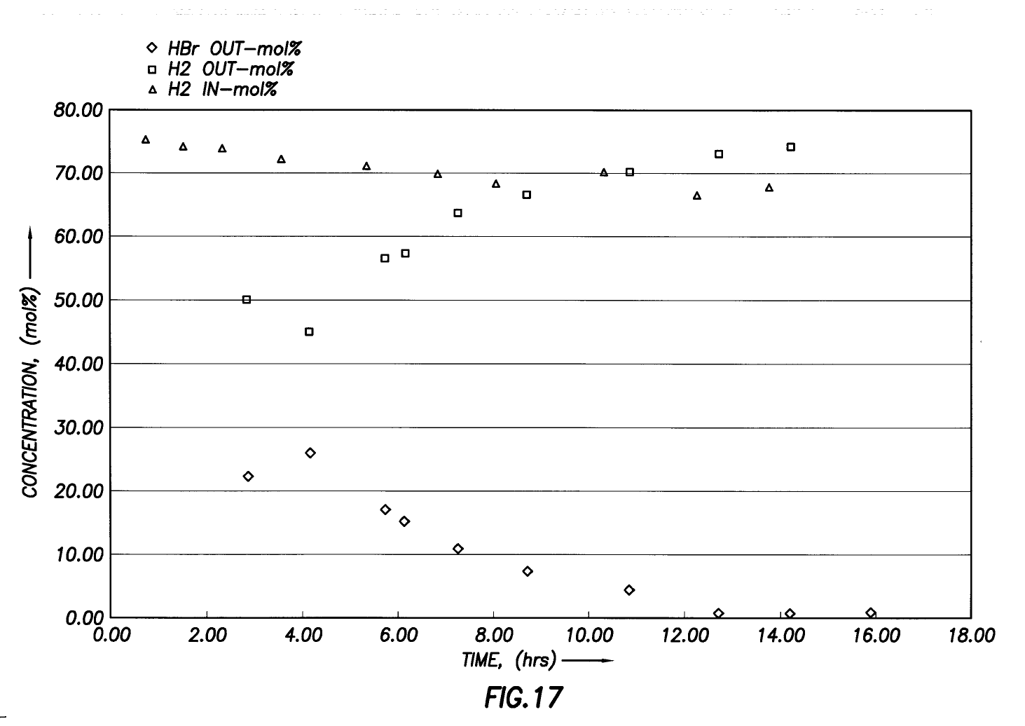

A mixture of di-brominated methane, methane, and hydrogen was reacted at 390° C. at 60 psig over a catalyst with a gas hourly space velocity (defined as the gas flow rate in standard liters per hour divided by the gross reactor-catalyst bed volume, including catalyst-bed porosity, in liters) of approximately 750 hr⁻¹. The catalyst comprised ferric bromide dispersed on a low-surface-area silica support. FIG. 15 is a graph illustrating the conversion di-brominated methane versus time. FIG. 16 is graph illustrating the concentration of di-brominated methane and mono-brominated methane in the streams entering and leaving the reactor. FIG. 17 is a graph illustrating the concentration of hydrogen bromide and hydrogen in the streams entering and leaving the reactor. As illustrated by this example, the conversion of di-brominated methane to mono-brominated methane may occur with near 100% selectively. In addition, it can also be inferred from these results that di-brominated methane is substantially more reactive with respect to hydrogenation over this catalyst than mono-brominated methane. Otherwise, the brominated methane would have been partially or totally converted to methane and HBr.

### EXAMPLE 2

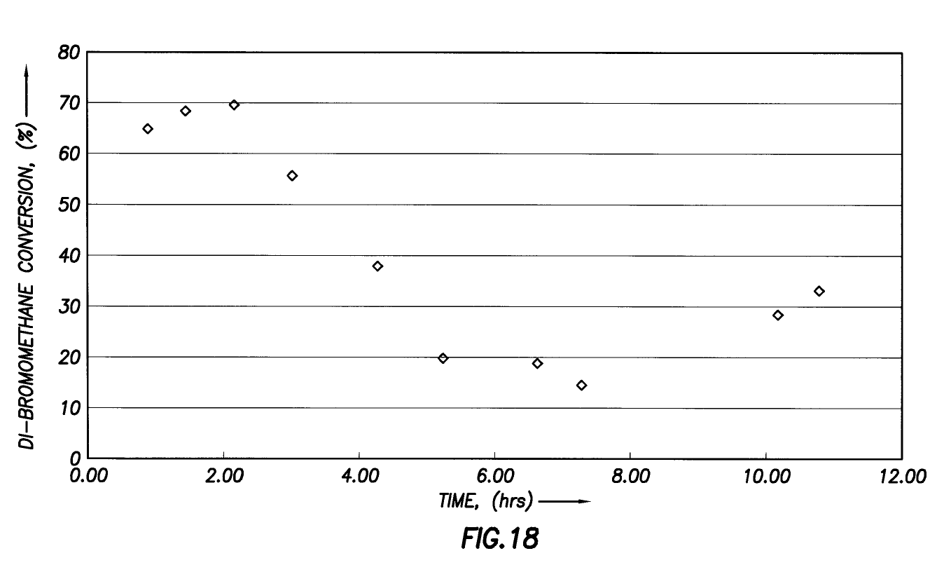

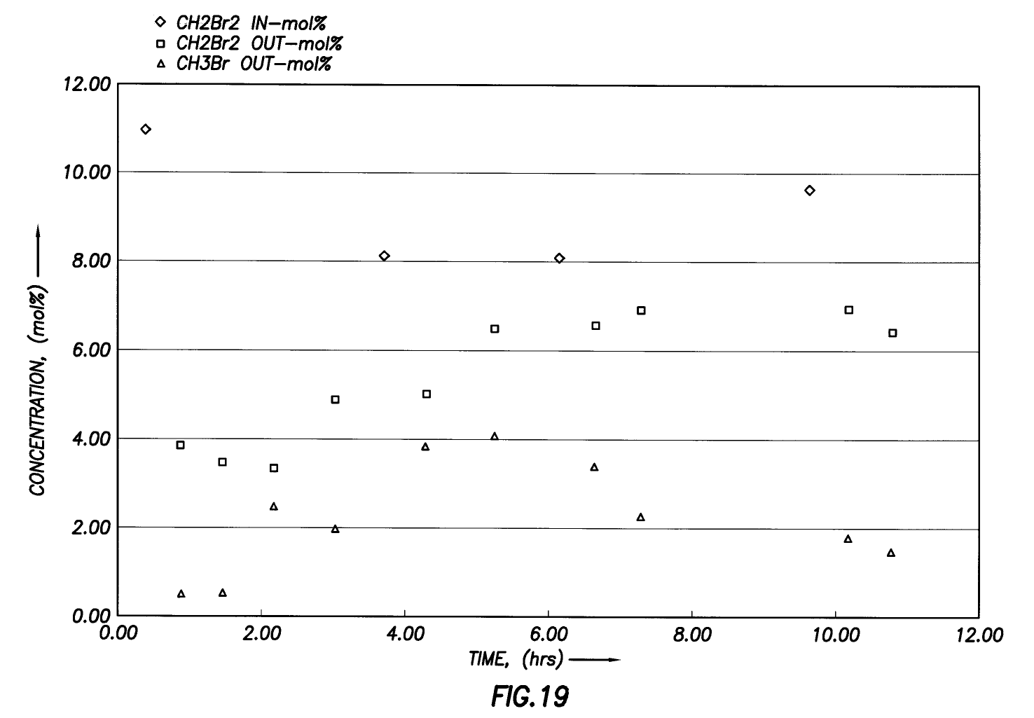

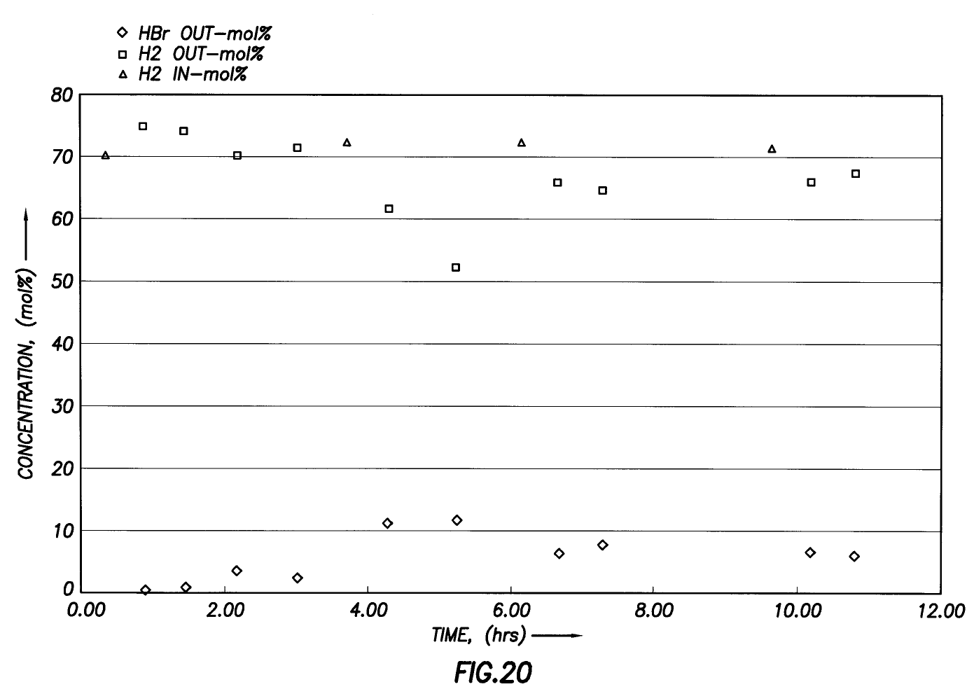

A mixture of di-brominated methane, methane, and hydrogen was reacted at 390° C. and 60 psig over a catalyst with a gas hourly space velocity of approximately 750 hr⁻¹. The catalyst comprised platinum dispersed on a low-surface-area silica support. FIG. 18 is a graph illustrating the conversion di-brominated methane versus time. FIG. 19 is graph illustrating the concentration of di-brominated methane and mono-brominated methane in the streams entering and leaving the reactor. FIG. 20 is a graph illustrating the concentration of hydrogen bromide and hydrogen in the streams entering and leaving the reactor. In addition, it can also be inferred from these results that di-brominated methane is substantially more reactive with respect to hydrogenation over this catalyst than mono-brominated methane. Otherwise, the brominated methane would have been partially or totally converted to methane and HBr.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified, and all such variations are considered within the scope and spirit of the present invention. In particular, every range of values (of the form, “from about a to about b,” or, equivalently, “from approximately a to b,” or, equivalently, “from approximately a-b”) disclosed herein is to be understood as referring to the power set (the set of all subsets) of the respective range of values, and set forth every range encompassed within the broader range of values. Moreover, the indefinite articles “a” or “an”, as used in the claims, are defined herein to mean one or more than one of the element that it introduces. Also, the terms in the claims have their plain, ordinary meaning unless otherwise explicitly and clearly defined by the patentee.

1. A method comprising:
reacting at least hydrogen and multi-brominated alkanes in the presence of a catalyst to form a hydrogenated stream comprising brominated alkanes having fewer bromine substituents than the multi-brominated alkanes reacted with the hydrogen.

2. The method of claim 1, wherein the multi-brominated alkanes comprise di-brominated methane, and wherein the brominated alkanes having fewer bromine substituents comprise mono-brominated methane.

3. The method of claim 1, wherein the catalyst comprises a catalyst capable of forming multiple thermally reversible complexes with bromine.

4. The method of claim 1, wherein the catalyst comprises a catalyst selected from the group consisting of iron oxide deposited on a support and platinum dispersed on a support.

5. A method comprising:
forming bromination products comprising brominated alkanes from bromination reactants comprising alkanes and bromine, wherein the brominated alkanes comprise mono-brominated alkanes and multi-brominated alkanes;
forming hydrogenation products comprising additional mono-brominated alkanes from hydrogenation reactants comprising hydrogen and at least a portion of the multi-brominated alkanes formed from the bromination reactants; and
forming synthesis products comprising hydrocarbons from synthesis reactants comprising reactant mono-brominated bromines, wherein the reactant mono-brominated bromines comprise at least a portion of the mono-brominated alkanes formed from the bromination reactants and at least a portion of the additional mono-brominated alkanes formed from the hydrogenation reactants.

6. The method of claim 5, wherein the multi-brominated alkanes present in the brominated alkanes comprise di-brominated methane.

7. The method of claim 5, wherein forming the bromination products comprises reacting at least the alkanes and the bromine in the presence of a catalyst.

8. The method of claim 5, wherein forming the hydrogenation products comprises reacting at least the hydrogen and the portion of the multi-brominated alkanes in the presence of a catalyst.

9. The method of claim 8, wherein the catalyst comprises a catalyst capable of forming multiple thermally reversible complexes with bromine.

10. The method of claim 8, wherein the catalyst comprises a catalyst selected from the group consisting of iron oxide deposited on a support and platinum dispersed on a support.

11. The method of claim 5, comprising reacting at least alkanes and steam to form produced hydrogen, wherein the hydrogen present in the hydrogenation reactants comprises the produced hydrogen.

12. The method of claim 5, comprising electrolyzing hydrogen bromide to form produced hydrogen, wherein the hydrogen present in the hydrogenation reactants comprises the produced hydrogen.

13. The method of claim 5, comprising electrolyzing a metal bromide salt to form produced hydrogen, wherein the hydrogen present in the hydrogenation reactants comprises the produced hydrogen.

14. The method of claim 5, wherein forming the synthesis products comprises reacting at least the reactant mono-brominated alkanes in the presence of a catalyst.

15. The method of claim 14, wherein the catalyst comprises a synthetic crystalline aluminosilicate oxide framework.

16. The method of claim 5, comprising recovering a liquid product stream comprising C5+ hydrocarbons, wherein the C5+ hydrocarbons are present in the hydrocarbons formed in the step of forming synthesis products.

17. The method of claim 5, comprising recovering a liquid product stream comprising olefins, wherein the olefins are present in the hydrocarbons formed in the step of forming synthesis products.

18. The method of claim 5, comprising:
separating hydrogen bromide from at least a portion of the hydrocarbons present in the synthesis products by dissolving the hydrogen bromide in water;
neutralizing at least a portion of the hydrogen bromide to form a metal bromide salt; and
oxidizing at least a portion of the metal bromide salt to form oxidation products comprising recovered bromine; and
recycling the recovered bromine formed in the oxidizing step, wherein the recovered bromide is used in forming additional brominated alkanes.

19. The method of claim 18, comprising:
electrolyzing another portion of the hydrogen bromide to form electrolysis products comprising produced hydrogen and additional recovered bromine,
recycling the produced hydrogen, and
recycling the additional recovered bromine.

20. The method of claim 18, comprising:
electrolyzing another portion of the metal bromide salt to form electrolysis products comprising produced hydrogen and additional recovered bromine,
recycling the produced hydrogen, and
recycling the additional recovered bromine.

21. The method of claim 5, comprising separating hydrogen bromide from at least a portion of the hydrocarbons present in the synthesis products, the separating comprising reacting the hydrogen bromide with a metal oxide to form a metal bromide;
oxidizing the metal bromide to form oxidation products comprising the metal oxide and recovered bromine; and
recycling the recovered bromine formed in the oxidizing step, wherein the recovered bromine is used in forming additional brominated alkanes.

22. The method of claim 21, comprising:
prior to the step of separating the hydrogen bromide, separating the synthesis products into a first synthesis product stream and a second synthesis product stream, wherein the first synthesis product stream comprises the portion of the hydrocarbons reacted with the metal oxide;
separating additional hydrogen bromide from the hydrocarbons present in the second synthesis product stream; and
electrolyzing at least a portion of the additional hydrogen bromide to form electrolysis products comprising produced hydrogen and additional recovered bromine;
recycling the produced hydrogen; and
recycling the recovered bromine.

23. The method of claim 21, comprising:
prior to the step of separating the hydrogen bromide, separating the synthesis products into a first synthesis product stream and a second synthesis product stream, wherein the first synthesis product stream comprises the portion of the hydrocarbons reacted with the metal oxide;
separating additional hydrogen bromide from the hydrocarbons present in the second synthesis product stream;
neutralizing the additional hydrogen bromide to form neutralization products comprising a metal bromide salt;
electrolyzing at least a portion of the additional metal bromide salt to form electrolysis products comprising produced hydrogen and additional recovered bromine;
recycling the produced hydrogen; and
recycling the additional recovered bromine.

24. The method of claim 5, comprising:
introducing the synthesis products into a product recovery unit, wherein the synthesis products further comprise hydrogen bromide and unreacted methane;
removing a liquid product stream comprising product hydrocarbons from the product recovery unit;
removing a stream comprising the hydrogen bromide and the unreacted methane from the product recovery unit;
electrolyzing at least a portion of the hydrogen bromide to form electrolysis products comprising recovered bromine and produced hydrogen;
recycling the recovered bromine; and
recycling at least a portion of the produced hydrogen.

25. The method of claim 5, comprising: operating at least one electrolysis cell used in the electrolyzing step in an air-depolarized mode, such that the electrolysis products further comprise water.

26. The method of claim 5, comprising separating the bromination products into a bypass stream and a hydrogenation feed stream comprising the hydrogenation reactants.

27. The method of claim 26, wherein the separating step comprises cooling the bromination products.

28. The method of claim 27, comprising:
heating the bypass stream; and
heating the hydrogenation feed stream.

29. A system comprising:
a bromination reactor configured to form bromination products comprising brominated alkanes from bromination reactants comprising alkanes and bromine, wherein the brominated alkanes comprise mono-brominated alkanes and multi-brominated alkanes;
a hydrogenation reactor in fluid communication with the bromination reactor and configured to form hydrogenation products comprising additional mono-brominated alkanes from hydrogenation reactants comprising hydrogen and at least a portion of the multi-brominated alkanes from the bromination reactor; and
a synthesis reactor in fluid communication with the hydrogenation reactor and configured to form synthesis products comprising hydrocarbons from synthesis reactants comprising reactant mono-brominated bromines, wherein the reactant mono-brominated bromines comprise at least a portion of the mono-brominated alkanes from the bromination reactor and at least a portion of the additional mono-brominated alkanes from the hydrogenation reactor.

30. The method of claim 5, wherein the alkanes are sourced from natural gas, coal-bed methane, regasified liquefied natural gas, gas derived from gas hydrates and/or chlathrates, gas derived from anerobic decomposition of organic matter or biomass, gas derived in the processing of tar sands, synthetically produced natural gas or alkanes, or mixtures of these sources.

31. The method of claim 5, wherein the alkanes are sourced from synthetically produced alkanes.

32. The method of claim 5, wherein the alkanes are sourced from synthetically produced natural gas.

33. The method of claim 5, wherein the alkanes are sourced from gas derived in the processing of tar sands.
